SEQUENCE LISTING (71 sequences)

SEQ ID NO: 1 - protein, 140 aa, synthetic construct
  REGION 1..140: note = Synthesized
MKLWLNWVFLLTLLHGIQCEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSS

SEQ ID NO: 2 - protein, 128 aa, synthetic construct
  REGION 1..128: note = Synthesized
MDFQVQIFSFLLISASVIVSRGEIVLTQSPATLSLSPGERATLSCSAISSVSYMYWYQQKPGQAPRLLIYDTSNLVSGVPARFSGSGSGTDYTLTISSLEPEDFAVYYCQQWNTYPYTFGGGTKVEIK

SEQ ID NO: 3 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 4 - protein, 15 aa, Homo sapiens
EPKSCDKTHTCPPCP

SEQ ID NO: 5 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 6 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, synthetic construct
  REGION 1..451: note = Synthesized
EVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, synthetic construct
  REGION 1..213: note = Synthesized
EIVLTQSPATLSLSPGERATLSCSAISSVSYMYWYQQKPGQAPRLLIYDTSNLVSGVPARFSGSGSGTDYTLTISSLEPEDFAVYYCQQWNTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 124 aa, Homo sapiens [sequence omitted]

SEQ ID NO: 11 - protein, 7 aa, synthetic construct
  REGION 1..7: note = Synthesized
SGGGGSG

SEQ ID NO: 12 - protein, 237 aa, synthetic construct
  REGION 1..237: note = Synthesized
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 13 - protein, 581 aa, synthetic construct
  REGION 1..581: note = Synthesized
EVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 14 - protein, 124 aa, Homo sapiens
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 15 - protein, 237 aa, synthetic construct
  REGION 1..237: note = Synthesized
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 16 - protein, 581 aa, synthetic construct
  REGION 1..581: note = Synthesized
EVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 17 - protein, 5 aa, synthetic construct
  REGION 1..5: note = Synthesized
DFYME

SEQ ID NO: 18 - protein, 19 aa, synthetic construct
  REGION 1..19: note = Synthesized
ASRNKANDYTTEYSASVKG

SEQ ID NO: 19 - protein, 10 aa, synthetic construct
  REGION 1..10: note = Synthesized
SYYRYDGMDY

SEQ ID NO: 20 - protein, 10 aa, synthetic construct
  REGION 1..10: note = Synthesized
SAISSVSYMY

SEQ ID NO: 21 - protein, 7 aa, synthetic construct
  REGION 1..7: note = Synthesized
DTSNLVS

SEQ ID NO: 22 - protein, 9 aa, synthetic construct
  REGION 1..9: note = Synthesized
QQWNTYPYT

SEQ ID NO: 23 - protein, 7 aa, synthetic construct
  REGION 1..7: note = Synthesized
NDNHWWN

SEQ ID NO: 24 - protein, 16 aa, synthetic construct
  REGION 1..16: note = Synthesized
YIDSSGSSDNNPSLKS

SEQ ID NO: 25 - protein, 11 aa, synthetic construct
  REGION 1..11: note = Synthesized
GGGRDYYGMDY

SEQ ID NO: 26 - protein, 15 aa, synthetic construct
  REGION 1..15: note = Synthesized
RASQSVSTSSYSYVH

SEQ ID NO: 27 - protein, 7 aa, synthetic construct
  REGION 1..7: note = Synthesized
YASNLES

SEQ ID NO: 28 - protein, 9 aa, synthetic construct
  REGION 1..9: note = Synthesized
QHSWDIPFT

SEQ ID NO: 29 - protein, 121 aa, synthetic construct
  REGION 1..121: note = Synthesized
QVQLQESGPGLVKPSQTLSLTCTVSGYSITNDNHWWNWIRQHPGKGLEWMGYIDSSGSSDNNPSLKSQITISRDTSKNQLSLKLSSVTAADTAVYYCARGGGRDYYGMDYWGQGTTVTVSS

SEQ ID NO: 30 - protein, 111 aa, synthetic construct
  REGION 1..111: note = Synthesized
DIVMTQSPDSLGVSLGERATINCRASQSVSTSSYSYVHWYQQKPGQPPKLLIKYASNLESGVPDRFSGSGSGTDFTLTISSLQAEDVAVYYCQHSWDIPFTFGQGTKLEIK

SEQ ID NO: 31 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 32 - protein, 238 aa, synthetic construct
  REGION 1..238: note = Synthesized
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 33 - protein, 451 aa, synthetic construct
  REGION 1..451: note = Synthesized
EVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 344 aa, synthetic construct
  REGION 1..344: note = Synthesized
EIVLTQSPATLSLSPGERATLSCSAISSVSYMYWYQQKPGQAPRLLIYDTSNLVSGVPARFSGSGSGTDYTLTISSLEPEDFAVYYCQQWNTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 35 - protein, 19 aa, synthetic construct
  REGION 1..19: note = Synthesized
MGWSWIFFFLLSGTASVLS

SEQ ID NO: 36 - protein, 11 aa, synthetic construct
  REGION 1..11: note = Synthesized
SGGGGSGGGGS

SEQ ID NO: 37 - protein, 121 aa, synthetic construct
  REGION 1..121: note = Synthesized
EVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSS

SEQ ID NO: 38 - protein, 275 aa, synthetic construct
  REGION 1..275: note = Synthesized
MGWSWIFFFLLSGTASVLSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDSGGGGSGGGGSEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSS

SEQ ID NO: 39 - protein, 586 aa, synthetic construct
  REGION 1..586: note = Synthesized
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDSGGGGSGGGGSEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 106 aa, synthetic construct
  REGION 1..106: note = Synthesized
EIVLTQSPATLSLSPGERATLSCSAISSVSYMYWYQQKPGQAPRLLIYDTSNLVSGVPARFSGSGSGTDYTLTISSLEPEDFAVYYCQQWNTYPYTFGGGTKVEIK

SEQ ID NO: 41 - protein, 241 aa, synthetic construct
  REGION 1..241: note = Synthesized
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDSGGGGSGGGGSEIVLTQSPATLSLSPGERATLSCSAISSVSYMYWYQQKPGQAPRLLIYDTSNLVSGVPARFSGSGSGTDYTLTISSLEPEDFAVYYCQQWNTYPYTFGGGTKVEIK

SEQ ID NO: 42 - protein, 348 aa, synthetic construct
  REGION 1..348: note = Synthesized
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDSGGGGSGGGGSEIVLTQSPATLSLSPGERATLSCSAISSVSYMYWYQQKPGQAPRLLIYDTSNLVSGVPARFSGSGSGTDYTLTISSLEPEDFAVYYCQQWNTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 208 aa, Homo sapiens [sequence omitted]

SEQ ID NO: 44 - protein, 8 aa, synthetic construct
  REGION 1..8: note = Synthesized
DYKDDDDK

SEQ ID NO: 45 - protein, 37 aa, synthetic construct
  REGION 1..37: note = Synthesized
PNKGSGTTSGTTRLLSGHTCFTLTGLLGTLVTMGLLT

SEQ ID NO: 46 - protein, 260 aa, synthetic construct
  REGION 1..260: note = Synthesized
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNTGGGDYKDDDDKGGGPNKGSGTTSGTTRLLSGHTCFTLTGLLGTLVTMGLLT

SEQ ID NO: 47 - protein, 220 aa, Homo sapiens
LKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDH

SEQ ID NO: 48 - protein, 272 aa, synthetic construct
  REGION 1..272: note = Synthesized
LKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHTGGGDYKDDDDKGGGPNKGSGTTSGTTRLLSGHTCFTLTGLLGTLVTMGLLT

SEQ ID NO: 49 - protein, 357 aa, synthetic construct
  REGION 1..357: note = Synthesized
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 16 aa, synthetic construct
  REGION 1..16: note = Synthesized
SGGGGSGGGGSGGGGS

SEQ ID NO: 51 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 52 - protein, 266 aa, synthetic construct
  REGION 1..266: note = Synthesized
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTTGGGDYKDDDDKGGGPNKGSGTTSGTTRLLSGHTCFTLTGLLGTLVTMGLLT

SEQ ID NO: 53 - protein, 451 aa, synthetic construct
  REGION 1..451: note = Synthesized
QVQLQESGPGLVKPSQTLSLTCTVSGYSITNDNHWWNWIRQHPGKGLEWMGYIDSSGSSDNNPSLKSQITISRDTSKNQLSLKLSSVTAADTAVYYCARGGGRDYYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 218 aa, synthetic construct
  REGION 1..218: note = Synthesized
DIVMTQSPDSLGVSLGERATINCRASQSVSTSSYSYVHWYQQKPGQPPKLLIKYASNLESGVPDRFSGSGSGTDFTLTISSLQAEDVAVYYCQHSWDIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 275 aa, synthetic construct
  REGION 1..275: note = Synthesized
MGWSWIFFFLLSGTASVLSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDSGGGGSGGGGSQVQLQESGPGLVKPSQTLSLTCTVSGYSITNDNHWWNWIRQHPGKGLEWMGYIDSSGSSDNNPSLKSQITISRDTSKNQLSLKLSSVTAADTAVYYCARGGGRDYYGMDYWGQGTTVTVSS

SEQ ID NO: 56 - protein, 586 aa, synthetic construct
  REGION 1..586: note = Synthesized
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDSGGGGSGGGGSQVQLQESGPGLVKPSQTLSLTCTVSGYSITNDNHWWNWIRQHPGKGLEWMGYIDSSGSSDNNPSLKSQITISRDTSKNQLSLKLSSVTAADTAVYYCARGGGRDYYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 581 aa, synthetic construct
  REGION 1..581: note = Synthesized
QVQLQESGPGLVKPSQTLSLTCTVSGYSITNDNHWWNWIRQHPGKGLEWMGYIDSSGSSDNNPSLKSQITISRDTSKNQLSLKLSSVTAADTAVYYCARGGGRDYYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 58 - protein, 138 aa, synthetic construct
  REGION 1..138: note = Synthesized
MDFGLIFFIVALLKGVQCEVQLVESGGGLVQPGGSLRLSCAASGFDFSRYWMSWVRQAPGKGLEWIGEINPDSSTINYAPSLKDRFTISRDNAKNSLYLQMNSLRAEDTAVYYCARHDYDEGIYFDYWGQGTTVTVSS

SEQ ID NO: 59 - protein, 5 aa, synthetic construct
  REGION 1..5: note = Synthesized
RYWMS

SEQ ID NO: 60 - protein, 17 aa, synthetic construct
  REGION 1..17: note = Synthesized
EINPDSSTINYAPSLKD

SEQ ID NO: 61 - protein, 11 aa, synthetic construct
  REGION 1..11: note = Synthesized
HDYDEGIYFDY

SEQ ID NO: 62 - protein, 128 aa, synthetic construct
  REGION 1..128: note = Synthesized
MDFQVQIFSFLLISASVIISRGDIQMTQSPSSLSASVGDRVTITCSASSRVIYMHWYQQKPGKAPKRWIYDTSKLASGVPSRFSGSGSGTEFTLTISSLQPEDFATYYCQQWSSNPPTFGGGTKVEIK

SEQ ID NO: 63 - protein, 10 aa, synthetic construct
  REGION 1..10: note = Synthesized
SASSRVIYMH

SEQ ID NO: 64 - protein, 7 aa, synthetic construct
  REGION 1..7: note = Synthesized
DTSKLAS

SEQ ID NO: 65 - protein, 9 aa, synthetic construct
  REGION 1..9: note = Synthesized
QQWSSNPPT

SEQ ID NO: 66 - protein, 450 aa, synthetic construct
  REGION 1..450: note = Synthesized
EVQLVESGGGLVQPGGSLRLSCAASGFDFSRYWMSWVRQAPGKGLEWIGEINPDSSTINYAPSLKDRFTISRDNAKNSLYLQMNSLRAEDTAVYYCARHDYDEGIYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 213 aa, synthetic construct
  REGION 1..213: note = Synthesized
DIQMTQSPSSLSASVGDRVTITCSASSRVIYMHWYQQKPGKAPKRWIYDTSKLASGVPSRFSGSGSGTEFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 120 aa, synthetic construct
  REGION 1..120: note = Synthesized
EVQLVESGGGLVQPGGSLRLSCAASGFDFSRYWMSWVRQAPGKGLEWIGEINPDSSTINYAPSLKDRFTISRDNAKNSLYLQMNSLRAEDTAVYYCARHDYDEGIYFDYWGQGTTVTVSS

SEQ ID NO: 69 - protein, 255 aa, synthetic construct
  REGION 1..255: note = Synthesized
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDSGGGGSGGGGSEVQLVESGGGLVQPGGSLRLSCAASGFDFSRYWMSWVRQAPGKGLEWIGEINPDSSTINYAPSLKDRFTISRDNAKNSLYLQMNSLRAEDTAVYYCARHDYDEGIYFDYWGQGTTVTVSS

SEQ ID NO: 70 - protein, 585 aa, synthetic construct
  REGION 1..585: note = Synthesized
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDSGGGGSGGGGSEVQLVESGGGLVQPGGSLRLSCAASGFDFSRYWMSWVRQAPGKGLEWIGEINPDSSTINYAPSLKDRFTISRDNAKNSLYLQMNSLRAEDTAVYYCARHDYDEGIYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 580 aa, synthetic construct
  REGION 1..580: note = Synthesized
EVQLVESGGGLVQPGGSLRLSCAASGFDFSRYWMSWVRQAPGKGLEWIGEINPDSSTINYAPSLKDRFTISRDNAKNSLYLQMNSLRAEDTAVYYCARHDYDEGIYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

(19) United States
(12) Patent Application Publication
(10) Pub. No.: US 2024/0317870 A1
(43) Pub. Date: Sep. 26, 2024

(54) BIFUNCTIONAL MOLECULES FOR TREATMENT OF IMMUNE DISORDERS

(71) Applicant: JN BIOSCIENCES LLC, MOUNTAIN VIEW, CA (US)

(72) Inventors: Naoya Tsurushita, Palo Alto, CA (US); J. Yun Tso, Menlo Park, CA (US)

(73) Assignee: JN BIOSCIENCES LLC, MOUNTAIN VIEW, CA (US)

(21) Appl. No.: 18/657,628

(22) Filed: May 7, 2024

Related U.S. Application Data
(63) Continuation of application No. 17/694,570, filed on Mar. 14, 2022, now Pat. No. 11,993,657.
(60) Provisional application No. 63/161,713, filed on Mar. 16, 2021.

(51) Int. Cl. C07K 16/28; A61K 47/68; A61P 37/06

(52) U.S. Cl. CPC C07K 16/2866; A61K 47/6811; A61K 47/6849; A61K 47/6889; A61P 37/06

(57) ABSTRACT

The invention provides bifunctional molecules including an antibody specifically binding to CD122 and an extracellular domain of CTLA-4. The bifunctional molecules specifically bind to CD122 and CTLA-4 ligands, CD80 and CD86 and inhibit their function in immune activation. The bifunctional molecules can inhibit interaction of CD122 with its ligands IL-2 and IL-15 and inhibit interaction of CD80 and CD86 with their counter-receptor, CD28. These bifunctional molecules can suppress Signals 2 and 3 of immune responses as a single therapeutic agent for treatment of immune disorders.

### CROSS-REFERENCE TO RELATED APPLICATION

The application is a continuation of U.S. Ser. No. 17/694,570 filed Mar. 14, 2024, which claims the benefit of U.S. 63/161,713, filed Mar. 16, 2021, incorporated by reference in its entirety for all purposes.

### SEQUENCE LISTING

The application includes sequences in an XML file named 610492SEQLST.XML. containing 97,229 bytes, and created May 7, 2024, incorporated by reference.

### BACKGROUND

Antigen-specific immune response is a complex biological process that is controlled by multiple layers of positive and negative regulators. T cells are initially stimulated through the T cell receptor (TCR) by the recognition of their cognate peptide antigen presented by major histocompatibility complex (MHC; also called HLA for human proteins) molecules on antigen-presenting cells (APCs). The initial interaction between TCR and MHC (or HLA) for T cell activation is referred to as Signal 1. Optimal T cell activation requires a second signal provided by costimulatory molecules such as CD28 and ICOS that belong to the CD28 superfamily (Signal 2). The immune system is further regulated positively by other costimulatory molecules such as CD40, OX40, GITR, CD27, HVEM and 4-1BB that belong to the TNF receptor superfamily and negatively regulated by checkpoint molecules such as PD-1, TIGIT, TIM-3, LAG-3, BTLA, VISTA, CD96 and CD112R. These costimulatory and checkpoint molecules are expressed in a cell type-and development stage-dependent manner to delicately control immune responses in the body. For reviews, see Pardoll, Nat. Rev. Cancer, 12: 252-264, 2012; Mahoney et al., Nat. Rev. Drug Discov. 14: 561-584, 2015; Shin et al., Curr. Opin. Immunol. 33: 23-35, 2015; Marquez-Rodas et al. Ann. Transl. Med. 3: 267, 2015; Mercier et al., Front. Immunol. 6: 418, 2015; Topalian et al., Cancer Cell 27: 450-461, 2015; Baumeister et al., Annu. Rev. Immunol. 34: 539-573, 2016; Ward-Kavanagh et al., Immunity 44: 1005-1019, 2016; Torphy et al., Int. J. Mol. Sci. 18: 2642, 2017.

CTLA-4 (cytotoxic T-lymphocyte-associated protein 4; also called CD152) is a negative regulator of immune responses. CTLA-4 binds to CD80 (also called B7-1) and CD86 (also called B7-2) expressed on the surface of APCs, interferes in the interaction of CD80 and CD86 with CD28 expressed on T cells, and blocks CD28-mediated costimulation of T cells. Abatacept is a recombinant protein comprising an extracellular domain of human CTLA-4 fused to the Fc region of human gamma-1 heavy chain (CTLA4-Ig). Abatacept was approved for marketing in the U.S. for treatment of adult rheumatoid arthritis, polyarticular juvenile idiopathic arthritis, and adult psoriatic arthritis. Belatacept, a successor of abatacept, is also a CTLA4-Ig fusion protein, which carries two amino acid substitutions in the CTLA-4 region, one from an alanine residue to a tyrosine residue at position 29 (A29Y) and another from a leucine residue to a glutamate residue at position 104 (L104E), to enhance the binding to CD80 and CD86 (Larsen et al., Am. J. Transplant. 5: 443-453, 2005; Oshima et al., Protein Eng. Des. Sel. 29: 159-167, 2016). Belatacept was approved for marketing in the U.S. for prevention of renal allograft rejection. For reviews of abatacept and belatacept, see Moreland et al., Nat. Rev. Drug Disc. 5: 185-186, 2006; Esensten et al., Immunity 44: 973-988, 2016; Perez et al., Transplantation 102: 1440-1452, 2008; Gallo et al., PLOS ONE 15: e0240335, 2020.

A variety of secreted proteins, such as cytokines and chemokines, are additionally involved in regulation of immune responses by promoting activation, differentiation, proliferation, maintenance, and suppression of certain subsets of immune cells. The action of cytokines on T cells is often referred to as Signal 3, which is the third mechanism required for activation, differentiation, and proliferation of T cells (Gutcher et al., J. Clin. Invest. 117: 1119-1127, 2007; Curtsinger et al., Curr. Opin. Immunol. 22: 333-340, 2010; Hurton et al., Proc. Natl. Acad. Sci. 113: E7788-E7797, 2016).

Interleukin-15 (IL-15) is a proinflammatory cytokine that is required for activation, proliferation, and maintenance of natural killer (NK) cells and CD8+ memory T cells. IL-15 is secreted mainly by APCs and captured at a high affinity by IL-15 receptor alpha subunit (also called CD215) expressed on the surface of the same or nearby cell. The IL-15/CD215 complex on the cell surface is then presented to the IL-15 receptor comprising CD122 (beta chain subunit of IL-2 and IL-15 receptors) and CD132 (common gamma chain) molecules on the surface of another cell and triggers intracellular signal transduction for induction of cellular functions (trans-presentation of IL-15). For reviews, see Waldmann et al., Annu. Rev. Immunol. 17: 19-49, 1999; Stonier et al., Immunol. Lett. 127: 85-92, 2010; Anthony et al., Research and Reports in Biology, 6: 25-37, 2015; Fehniger, J. Immunol. 202: 3125-3126, 2019; Yang et al., Cancers 12: 3586, 2020.

Recent studies have shown that a variety of autoimmune diseases are caused by self-reacting D8+ memory T cells, some of them residing in tissues (tissue-resident memory T cells; Topham et al., Front. Immunol. 9: Article 515, 2018). It has been reported that anti-CD122 monoclonal antibodies that block the interaction between IL-15 and CD122 (thus IL-15 receptor) is therapeutically efficacious in mouse models of vitiligo (Richmond et al., Sci. Trans. Med. 10: eaam7710, 2018), alopecia areata (Xing et al., Nat. Med. 20: 1043-1049, 2014), type 1 diabetes (Chen et al., Proc. Natl. Acad. Sci. 110: 13534-13539, 2013; Yuan et al. JCI Insight 3:e96600, 2018), celiac disease (Yokoyama et al., Proc. Natl. Acad. Sci. 106: 15849-15854, 2009), non-alcoholic steatohepatitis (NASH) (Dudek et al., Nature 592: 444-449, 2021) and skin allograft rejection (Mathews et al., J. Clin. Invest. 128: 4557-4572, 2018). The role of CD8+ memory T cells is also implicated in pathogenesis of multiple sclerosis (Fransen et al., Brain 143: 1714-1730, 2020), polymyositis (Kamiya et al., Rheumatol. 59: 224-232, 2020), allergic dermatitis (Gadsbøll et al., J. Invest. Dermatol. 140: 806-815, 2020), primary biliary cirrhosis (Yang et al., J. Immunol. 186: 1259-1267, 2011), Behçet's disease (Yu et al., Clin. Exp. Immunol. 137: 437-443, 2004), and ulcerative colitis (Nemeth et al., Cureus 9: e1177, 2017; Boland et al., Sci. Immunol. 5: eabb4432, 2020).

### SUMMARY OF THE CLAIMED INVENTION

The invention provides a bifunctional molecule comprising an antibody that binds to CD122 and inhibits its interaction with IL-2 and IL-15, wherein the antibody is linked at an N-or C-terminus to an extracellular domain of CTLA-4.

Optionally the antibody comprises a heavy chain comprising a mature heavy chain variable region linked to a heavy chain constant region linked at its C-terminus to the extracellular domain of CTLA-4, and a light chain comprising a mature light chain variable region linked to a light chain constant region. Optionally, the antibody is linked to the extracellular domain of CTLA-4 via the C-terminus of the heavy chain constant region or the C-terminus of the light chain constant region. Optionally, the antibody is linked to the extracellular domain of CTLA-4 via the N-terminus of the mature heavy chain variable region or the N-terminus of the mature light chain variable region. Optionally, the heavy or light chain is linked to the extracellular domain of CTLA-4 via a polypeptide linker. Optionally, the polypeptide linker comprises the amino acid sequence of SEQ ID NO: 11, 36 or 50. Optionally, the bifunctional molecule is in the form of a heterodimer comprising two copies of the heavy chain and two copies of the light chain. Optionally, the antibody comprises three heavy chain CDRs comprising SEQ ID NOS: 17-19 respectively and three light chain CDRs comprising SEQ ID NOS:20-22 respectively. Optionally, the antibody comprises a mature heavy chain variable region comprising SEQ ID NO:37 and a light chain variable region comprising SEQ ID NO:40. Optionally, the heavy chain comprises SEQ ID NO: 8 provided the C-terminal lysine can be omitted, and the light chain comprises SEQ ID NO:9. Optionally, the mature heavy chain is linked to the CTLA-4 extracellular domain as a chain comprising SEQ ID NO:13 or 16 and the mature light chain comprises SEQ ID NO:9. Optionally, the mature light chain is linked to the CTLA-4 extracellular domain as a chain comprising SEQ ID NO:34 and the mature heavy chain comprises SEQ ID NO:8 or 33 provided the C-terminal lysine can be omitted. Optionally, the mature heavy chain is linked to the CTLA-4 extracellular domain as a chain comprising SEQ ID NO:39 provided the C-terminal lysine can be omitted, and the mature light chain comprises SEQ ID NO:9. Optionally the mature light chain is linked to the CTLA-4 extracellular domain as a chain comprising SEQ ID NO:42, and the mature heavy chain comprises SEQ ID NO: 8 or 33 provided the C-terminal lysine can be omitted.

Optionally, the antibody comprises three heavy chain CDRs comprising SEQ ID NOS:23-25 respectively, and three light chain CDRs comprising SEQ ID NOS:26-28 respectively. Optionally, the antibody comprises a mature heavy chain variable region comprising SEQ ID NO:29 and a mature light chain variable region comprising SEQ ID NO:30. Optionally, the mature heavy chain comprises SEQ ID NO:53 provided the C-terminal lysine can be omitted and the mature light chain comprises SEQ ID NO:54. Optionally, the mature heavy chain is linked to the CTLA-4 extracellular domain as a chain comprising SEQ ID NO:57 and the mature light chain comprises SEQ ID NO:54. Optionally, the mature heavy chain is linked to the CLTA-4 extracellular domain as a chain comprising SEQ ID NO:56 provided the C-terminal lysine can be omitted, and the mature light chain comprises SEQ ID NO:54.

Optionally, the antibody comprises three heavy chain CDRs comprising SEQ ID NOS: 59-61 respectively and three light chain CDRs comprising SEQ ID NOS:63-65 respectively. Optionally, the mature heavy chain variable region comprises SEQ ID NO:58 and the mature light chain variable region comprises SEQ ID NO:62. Optionally, the mature heavy chain comprises SEQ ID NO:66 provided the C-terminal lysine can be omitted, and the mature light chain comprises SEQ ID NO:67. Optionally, the mature heavy chain is linked to the CTLA-4 extracellular domain as a chain comprising SEQ ID NO:71 and the mature light chain comprises SEQ ID NO:67. Optionally, the mature heavy chain is linked to the CTLA-4 extracellular domain as a chain comprising SEQ ID NO:70 provided the C-terminal lysine can be omitted, and the mature light chain comprises SEQ ID NO:67.

Optionally, the antibody has human IgG1 kappa isotype. Optionally, the heavy chain constant region has one or more mutations to reduce effector function. Optionally, the mutations are L234A and L235A. Optionally, the extracellular domain is of a human CTLA-4. Optionally, the extracellular domain of CTLA-4 comprises SEQ ID NO:10. Optionally, the extracellular domain of CTLA-4 comprises SEQ ID NO:14.

The invention further provides a pharmaceutical composition comprising a bifunctional molecule of any preceding claim and a pharmaceutically acceptable carrier.

The invention further provides a method of treating a subject having an immune disorder, comprising administering an effective regime of a bifunctional molecule of any preceding claim to the subject. Optionally, the immune disorder is any of vitiligo, alopecia areata, type 1 diabetes, celiac disease, non-alcoholic steatohepatitis, skin allograft rejection, multiple sclerosis, polymyositis, allergic dermatitis, primary biliary cirrhosis, Behçet's disease, and ulcerative colitis. Optionally, the immune disorder is graft rejection or graft versus host disease. Optionally, the immune disorder is an autoimmune disease.

The invention further provides an antibody against human CD122, wherein the antibody comprises three heavy chain CDRs comprising SEQ ID NOS: 59-61 respectively and three light chain CDRs comprising SEQ ID NOS. 63-65 respectively. Optionally, the antibody comprises a mature heavy chain variable region comprising SEQ ID NO:58 and a mature light chain variable region comprising SEQ ID NO:62. Optionally, the mature heavy chain comprises SEQ ID NO:66 provided the C-terminal lysine can be omitted, and the mature light chain comprises SEQ ID NO:67.

### BRIEF DESCRIPTION OF THE FIGURES

FIGS. 1A, B: Schematic structure of the expression vectors pHuABC2p (A), and pHC624 and pHC625 (B).

FIG. 2: Schematic structure of bifunctional molecules HuABC2-CTLA4 and HuABC2-CTLA4YE.

FIG. 3: Binding of HuABC2, HuABC2-CTLA4 and HuABC2-CTLA4YE to CD122. Average absorbance levels of duplicate analysis are shown with standard deviation bars.

FIG. 4: Binding of HuABC2, HuABC2-CTLA4 and HuABC2-CTLA4YE to CD80. Average absorbance levels of duplicate analysis are shown with standard deviation bars.

FIG. 5: Binding of HuABC2, HuABC2-CTLA4 and HuABC2-CTLA4YE to CD86. Average absorbance levels of duplicate analysis are shown with standard deviation bars.

FIGS. 6A, B, C: Schematic structures of the expression vectors pHC623 (A), pHC640 (B), and pHC641 (C).

FIGS. 7A, B, C: Schematic structures of bifunctional molecules HuABC2-Ck-CTLA4YE (A), HuABC2-CTLA4YE-VH (B), and HuABC2-CTLA4YE-Vk (C).

FIGS. 8A, B, C: Schematic structures of the expression vectors pFCm553 (A), pFCm554 (B), and pFCm148 (C).

FIGS. 9A, B, C: Flow cytometry analysis of the binding of bifunctional molecules to CHO-K1/CD80 cells (A), CHO-K1/CD86 cells (B), and Ramos cells (C).

FIG. 10. Flow cytometry analysis of the binding of bifunctional molecules to NS0/CD122 cells

FIG. 11: Proliferation of human T cells in the mixed lymphocyte reaction.

FIG. 12: Expression of IFN-γ in the mixed lymphocyte reaction.

FIGS. 13A, B: Flow cytometry analysis of the binding of bifunctional molecules to CHO-K1/CD80 cells (A), and CHO-K1/CD86 cells (B).

### DEFINITIONS

Bifunctional molecules or other biological entities are typically provided in isolated form. This means that a biological entity is typically at least 50% w/w pure of interfering proteins and other contaminants arising from its production or purification but does not exclude the possibility that the entity is combined with an excess of pharmaceutically acceptable carrier(s) or other vehicle intended to facilitate its use. Sometimes entities are at least 60, 70, 80, 90, 95 or 99% w/w pure of interfering proteins and contaminants from production or purification. Often an isolated entity the predominant macromolecular species remaining after its purification.

Specific binding of a bifunctional molecule to a target protein, such as CD122, CD80 or CD86 means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a molecule binds one and only one target. Bifunctional molecules bind to multiple targets.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector functions.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, which in a natural antibody are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chain variable regions comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, MD, 1987 and 1991), or Chothia & Lesk, J. Mol. Biol. 196: 901-917 (1987); Chothia et al., Nature 342: 878-883 (1989). Kabat also provides a widely used numbering convention (Kabat numbering) in which corresponding residues between different heavy chains or between different light chains are assigned the same number.

The term “antibody” includes intact antibodies and binding fragments thereof. Typically, fragments compete with the intact antibody from which they were derived for specific binding to the target including separate heavy chains, light chains Fab, Fab′, F(ab′)2, F(ab)c, Dabs, nanobodies, and Fv. Fragments can be produced by recombinant DNA techniques, or by enzymatic or chemical separation of intact immunoglobulins.

The term “epitope” refers to a site on an antigen to which an antibody or bifunctional molecule binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50: 1495, 1990). A test antibody competes with a reference antibody if an excess of a test antibody (e.g., at least 2×, 5×, 10×, 20× or 100×) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “subject” includes human and other mammalian subjects that receive either prophylactic or therapeutic treatment. Unless otherwise apparent from the context, it also includes animal models used for testing such treatments.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. For non-antibody sequences, sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the longer of (a) entire length of the shorter of the two sequences being compared, or (b) at least 25 contiguous nucleotides.

When a value is characterized as being within a specified factor of a reference value, the specification should be understood as alternatively disclosing values higher or lower than the reference value within the factor.

Compositions or methods “comprising” one or more recited elements may include other elements not specifically recited. For example, a composition that comprises a bifunctional molecule may contain the bifunctional molecule alone or in combination with other ingredients.

### DETAILED DESCRIPTION

### I. General

The invention provides bifunctional molecules including an antibody specifically binding to CD122 and an extracellular domain of CTLA-4. The bifunctional molecules specifically bind to CD122 and CTLA-4 ligands, CD80 and CD86 and inhibit their function in immune activation. The bifunctional molecules can inhibit interaction of CD122 with its ligands IL-2 and IL-15 and inhibit interaction of CD80 and CD86 with their counter-receptor, CD28. These bifunctional molecules can suppress Signals 2 and 3 of immune responses as a single therapeutic agent for treatment of immune disorders.

Exemplary anti-CD122 antibodies for inclusion in bifunctional molecules are HuABC2 and HuABC101, which are humanized anti-CD122 IgG1/kappa monoclonal antibodies which bind to CD122 and block the interaction of CD122 with IL-2 and IL-15 (U.S. Pat. No. 9,028,830). Another exemplary anti-CD122 antibody for inclusion in bifunctional molecules is HuABC77, which is a humanized anti-CD122 IgG1/kappa monoclonal antibody which binds to CD122 and blocks the interaction of CD122 with IL-2 and IL-15. The CDRs of HuABC77 are SEQ ID NO:S 59-61 for the heavy chain and SEQ ID NOS: 63-65 for the light chain. HuABC2, which can be regarded as a Signal 3 blocker in the immune system, functions as a suppressor of NK cells and CD8+ memory T cells. HuABC2 was therapeutically efficacious for prevention of renal allograft rejection in non-human primates (Landolfi et al., Abstract No. 185, American Transplant Congress, 2013; Mathews et al. supra).

### II. CD122 and Antibodies Thereto

Unless otherwise indicated CD122 means human CD122. An exemplary human CD122 sequence is assigned Swiss Prot accession number P14784. The mature sequence of human CD122 without signal peptide is assigned SEQ ID NO:31. Known natural allelic variants thereof include 10 L-→V: dbSNP rs57770674, 83 S->F: dbSNP rs2228143, and 391 D→E: dbSNP rs228942. The complete human CD122 sequence has 551 amino acids of which amino acids 1-26 are a signal peptide. Approximately residues 27-240 constitute an extracellular domain. Approximately residues 241-265 constitute a transmembrane domain and approximately residues 266-551 constitute a cytoplasmic domain. Antibodies and bifunctional molecules used in the invention bind to epitopes within the extracellular domain of CD122.

CD122 forms receptors in combination with CD132, CD25 and/or IL-15Rα (also called CD215). In brief, the combination of CD122 and CD132 constitutes a receptor with intermediate affinity for IL-2 and IL-15. The combination of CD122, CD132 and CD25 constitutes a receptor with high affinity for IL-2 and the combination of CD122, CD132 and IL-15Rα constitutes a receptor with high affinity for IL-15.

Unless otherwise indicated CD132, CD25 and/or IL-15Rα refer to the human forms of these proteins. An exemplary human sequence for human CD132 is designated Swiss Prot P31785, which is a protein of 369 amino acids of which approximately residues 1-22 are a signal peptide, 23-262 are an extracellular domain, 263-283 are a transmembrane domain and 284-369 are a cytoplasmic domain.

An exemplary human sequence for CD25 is designated Swiss Prot P01589, which is an amino acid sequence of 272 residues of which approximately residues 1-21 are a signal peptide, residues 22-240 are an extracellular domain, residues 241-259 are a transmembrane region and residues 260-272 are a cytoplasmic domain.

An exemplary human sequence for IL-15Rα is designated Swiss Prot Q13261, which is a protein of 267 residues of which approximately 1-30 are a signal peptide, 31-205 are an extracellular domain, 206-228 are a transmembrane region, and 229-267 are a cytoplasmic domain.

Unless otherwise apparent from the context, reference to one of the above receptors means at least the extracellular domain of the protein and usually the complete protein other than a cleavable signal peptide.

The above receptors bind to IL-2 and/or IL-15, the latter of which may be presented in cis or in trans. Unless otherwise apparent from the context, these molecules refer to the human forms of these proteins. An exemplary sequence for human IL-2 is designated Swiss Prot P60568, which is a protein sequence of 153 amino acids of which amino acids 1-20 are a signal peptide. An exemplary sequence for human IL-15 is designated Swiss Prot P40933, which is a protein of 162 amino acids of which amino acids 1-29 are a signal peptide. Unless other apparent from the context, reference to IL-2 or IL-15 means the mature protein from which the signal sequence has been removed.

The invention uses monoclonal antibodies binding to epitopes within the CD122 protein. Antibodies designated ABC2 and ABC101 are two such exemplary mouse antibodies. Both ABC2 and ABC101 specifically bind to human CD122. ABC2 and ABC101 are further characterized by specific binding to cynomolgus CD122 and lack of significant binding to mouse and dog CD122. Binding to CD122 can be demonstrated to CD122 alone and/or any of the receptors incorporating CD122 discussed above. Such monoclonal antibodies are characterized in that as a single agent such a monoclonal antibody has a capacity to substantially inhibit receptors incorporating CD122 from binding to and signaling for cell proliferation in response to both IL-2 and IL-15.

Preferred antibodies and bifunctional molecules incorporating such antibodies inhibit binding of both low and high affinity receptors incorporating CD122 to both IL-2 and IL-15 and can inhibit IL-15 binding both in cis and in trans.

Inhibition may be demonstrated in a binding assay in which a receptor incorporating CD122 is expressed on cells and binds to IL-2 or IL-15 in the presence of an antibody or bifunctional molecule incorporating an antibody being tested. Alternatively, or additionally, inhibition can be demonstrated by expressing a receptor incorporating CD122 on appropriate cells in the presence of IL-2 or IL-15 and assessing an effect of a monoclonal antibody or bifunctional molecule incorporating an antibody on IL-2 or IL-15-mediated proliferation of the cells. Inhibition of IL-15 can be tested in cis or in trans in such formats. Exemplary assay formats for showing inhibition are described in U.S. Pat. No. 9,028,830. Optionally, inhibition of a test antibody or bifunctional molecule can be demonstrated in comparison to an irrelevant control antibody not binding to CD122 or its receptor co-components or to IL-2 and IL-15, or to vehicle lacking any antibody.

Substantial inhibition means an inhibition of at least, 25, 30, 40, 50, or 75%, (e.g., 25-75% or 30-70%) of binding, cell proliferation and/or other functional activity mediated by IL-2 or IL-15. Inhibition is usually demonstrated when the antibody or bifunctional molecule is at no more than 100-fold molar excess (e.g., 2-50 or 2-10 or 2-5 fold molar excess) with respect to the IL-2 or IL-15 ligand. For functional assays, IL-2 or IL-15 is typically present at the minimum level needed to stimulate full functional activity in the absence of antibody or bifunctional molecule under test. Antibodies or bifunctional molecules are usually present at a concentration between 50 nM and 5 μM. Preferred antibodies or bifunctional molecules show inhibition of at least 40% of IL-2 interaction with low or high affinity receptors (or both), and at least 40% for IL-15 presented in cis and in trans. The extent of inhibition can also be quantified by the antibody or bifunctional molecule concentration required for 50% inhibition of proliferation (IC50) mediated by trans-presentation of IL-15 to TF-1β cells as described in Examples 2 and 8 of U.S. Pat. No. 9,028,830. An antibody or bifunctional molecule concentration less than 2 μg/ml and preferably less 1 μg/ml, for example about 0.1 to 1 or 0.5 to 1 μg/ml is preferred. Additionally, the antibody or bifunctional molecule concentration required for 50% inhibition of IL-2-mediated TF-1αβ proliferation to cells bearing a high affinity IL-2 receptor as described in Example 8 of U.S. Pat. No. 9,028,830 is preferably less than 100 or 50 μg/ml, for example 10-50 μg/ml.

As is evident from these results the IC50 of an antibody or bifunctional molecule of the invention depends on the nature of the interaction being inhibited. For cells that have the high affinity IL-2 receptor, it is much more difficult for an anti-CD122 antibody or bifunctional molecule to inhibit IL-2 (i.e., higher concentration of the same antibody or bifunctional molecule needed) than to inhibit IL-15 trans-presentation. For cells that have only the intermediate affinity IL-2 receptor, it is harder to inhibit (i.e., higher concentration of the same antibody or bifunctional molecule needed) IL-15 trans-presentation than to inhibit soluble IL-2 or IL-15.

Some antibodies bind to the same or overlapping epitope as an antibody designated ABC2 or ABC101. ABC2 and its humanized versions is a monoclonal antibody that binds to CD122 and blocks the interaction of CD122 with IL-2 and IL-15, and functions as an immune suppressor of NK cells and CD8+ memory T cells. ABC2 and its humanized versions can therefore be regarded as a Signal 3 blocker in the immune system.

The sequences of the heavy and light chain CDRs of ABC2 are designated SEQ ID NOS: 17-19 and the light chain CDRs as defined by Kabat are designated SEQ ID NOS:20-22. The heavy and light chain CDRs of ABC101 as defined by Kabat are designated SEQ ID NOS:23-25 and 26-28 respectively. Full sequences of mouse and humanized versions are provided in U.S. Pat. No. 9,028,830. Other antibodies having such a binding specificity can be produced by immunizing mice, rats, hamsters, rabbits, or other animal species with CD122 or a portion thereof including the desired epitope, and screening resulting antibodies for binding to CD122, optionally in competition with ABC2 or ABC101. Antibodies identified by such assays can then be screened for ability to inhibit both IL-2 and IL-15 interactions as described in the examples, or otherwise. Antibodies can also be screened against mutagenized forms of the CD122 antigen to identify an antibody showing the same or similar binding profile to collection of mutational changes as ABC2 or ABC101. The mutations can be systematic replacement substitution with alanine (or serine if an alanine is present already) one residue at a time, or more broadly spaced intervals, throughout the extracellular domain of CD122 antibody or through a section thereof in which an epitope is known to reside.

Mutations at residues 42, 43 and 65 of CD122 inhibit specific binding of ABC2 to CD122 (e.g., <10% binding of a positive control anti-FLAG antibody as described as the examples of U.S. Pat. No. 9,028,830). Likewise, mutations at residues 65, 70 and 133 of SEQ ID NO:31 inhibit specific binding of ABC101 to CD122. Because relatively few residues affect binding and the residues are spaced more broadly than a typical linear epitope (e.g., 3-20 contiguous amino acids), these results provide an indication that ABC101 and possibly ABC2 binds to a conformational epitope. Alternatively, one or more of the residues affecting binding may do so allosterically without direct contact with the antibody. The observation that mutagenesis of residue 65 with threonine but not alanine inhibits binding provides an indication that this residue may be affecting binding allosterically. Other antibodies binding to an epitope including one or more of residues 42, 43, 65, 70 and 133 of CD122, and particularly to an epitope including one or more of residues 42, 65 and 133, are likely to share useful inhibitory properties with ABC2 and ABC101. Thus, antibodies whose specific binding is inhibited by mutagenesis of one or more or residues 42, 43 and 65 and particularly residues 42 and 65 of CD122 are likely to share similar properties to ABC2. Some such antibodies bind to an epitope that includes or consists of residue 42, 43 and/or 65 of CD122. Some such antibodies bind to an epitope that includes or consists of residues 42 and 43 and in which residue 65 affects binding of the antibody allosterically. The epitope can be linear, such as an epitope (e.g., 2-5, 3-5, 3-10, 3-15, 3-20, 5-10, 5-15 or 5-20 contiguous amino acids) including or consisting of 1, 2, or all 3 of the specified amino acids (42, 43 and 65) or be conformational including or consisting of 1, 2, or all 3 of the specified amino acids. Antibodies whose specific binding is inhibited by mutagenesis at 1, 2 or all 3 of residues 65, 70 and 133 of CD122 are likely to show similar inhibitory properties to ABC101. Some such antibodies bind to an epitope that includes or consists of residue 65 or 133 of CD122 or both. Such an epitope can be linear such as an epitope (e.g., 2-5, 3-5, 3-10, 3-15, 3-20, 5-10, 5-15 or 5-20 contiguous amino acids) including or consisting of 1, 2 or all 3 of the specified amino acids (65, 70, and 133) or be conformational including or consisting of 1, 2 or all 3 of the specified amino acids. Some antibodies of the invention specifically bind to CD122 without such binding being substantially inhibited (e.g., binding at least 50% of a positive control antibody as described in the Examples of U.S. Pat. No. 9,028,830) by substitution of residue 39 or 41 of SEQ ID NO:31 with alanine. Any of the above described antibody types includes antibodies that specifically bind to an epitope not including residue 39 or 41 of CD122 or, put another way, whose binding to CD122 is not detectably inhibited by substitution of these residues (binding above 50% of positive control anti-FLAG antibody as described in the examples).

Antibodies binding to an epitope that includes one or more specified residues can be generated by immunizing with a fragment of CD122 that includes these one or more residues. The fragment can for example have no more than 100, 50, 25, 10 or 5 contiguous amino acids from CD122. Such fragments usually have at least 5, 6, 7, 8 or 9 contiguous residues of SEQ ID NO:31. The fragments can be linked to a carrier that helps elicit an antibody response to the fragment and/or be combined with an adjuvant that helps elicit such a response. Alternatively, antibodies binding to a desired residue can be obtained by immunizing with a full-length CD122 (other than signal sequence) or full-length extracellular domain (other than signal sequence) of fibronectin III domain 1 and/or 2. Such antibodies can then be screened for differential binding to hybrids of human and mouse CD122 or differential binding to wildtype CD122 compared with mutants of specified residues. The screen against hybrids of human and mouse CD122 maps antibody binding to certain domains within CD122, such as fibronectin III domain 1 and/or 2. The screen against mutants more precisely defines the binding specificity to allow identification of antibodies whose binding is inhibited by mutagenesis of residues 43, 43, 65, 70 and/or 133 and which are likely to share inhibitor properties of the exemplified antibodies.

Antibodies having the binding specificity of a selected murine antibody (e.g., ABC2 or ABC101) can also be produced using a variant of the phage display method. See Winter, WO 92/20791. This method is particularly suitable for producing human antibodies. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions can for example be obtained from a library of rearranged human heavy chain variable regions. A phage showing strong specific binding for CD122 (e.g., at least 10⁸ and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for CD122 are selected. The resulting antibodies usually have the same or similar epitope specificity as the murine starting material.

Other antibodies can be obtained by mutagenesis of cDNA encoding the heavy and light chains of an exemplary antibody, such as ABC2 or ABC101 and their humanized forms. Monoclonal antibodies that are at least 90%, 95% or 99% identical to ABC2 or ABC101 or humanized forms in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Monoclonal antibodies having at least one and preferably all six CDR(s) as defined by Kabat that are 90%, 95%, 99% or 100% identical to corresponding CDRs of ABC2 or ABC101 are also included.

The production of other non-human monoclonal antibodies, e.g., murine, guinea pig, primate, rabbit or rat, against CD122 can be accomplished by, for example, immunizing the animal with CD122 or a fragment thereof, or cells bearing CD122, optionally co-expressed with its co-receptor proteins as discussed above. See Harlow & Lane, Antibodies, A Laboratory Manual (CSHP NY, 1988) (incorporated by reference for all purposes). Such an immunogen can be obtained from a natural source, by peptide synthesis or by recombinant expression. Optionally, the immunogen can be administered fused or otherwise complexed with a carrier protein. Optionally, the immunogen can be administered with an adjuvant. Several types of adjuvant can be used as described below. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to CD122. Optionally, antibodies are further screened for binding to a specific region of CD122. Such screening can be accomplished by determining binding of an antibody to a collection of deletion mutants of CD122 and determining which deletion mutants bind to the antibody. Binding can be assessed, for example, by Western blot, FACS or ELISA.

A humanized antibody is a genetically engineered antibody in which the CDRs from a non-human “donor” antibody are grafted into human “acceptor” antibody sequences (see, e.g., Queen, U.S. Pat. No. 5,530,101 and 5,585,089; Winter, U.S. Pat. No. 5,225,539; Carter, U.S. Pat. No. 6,407,213; Adair, U.S. Pat. No. 5,859,205; Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a non-human (e.g., mouse) antibody, they can also be made with less than all CDRs (e.g., at least 3, 4, or 5) CDRs from a non-human antibody (e.g., Pascalis et al., J. Immunol. 169: 3076, 2002; Vajdos et al., Journal of Molecular Biology, 320: 415-428, 2002; Iwahashi et al., Mol. Immunol. 36: 1079-1091, 1999; Tamura et al, Journal of Immunology, 164: 1432-1441, 2000).

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196: 901, 1987), by molecular modeling and/or empirically, or as described in Gonzales et al., Mol. Immunol. 41: 863, 2004. In such humanized antibodies at positions in which one or more donor CDR residues is absent or in which an entire donor CR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain.

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a non-human variable region framework residue and a selected human variable region framework residue, the human framework amino acid can be substituted by the equivalent framework amino acid from the non-human antibody when it is reasonably expected that the amino acid:
(1) noncovalently binds antigen directly,
(2) is adjacent to a CDR region,
(3) otherwise interacts with a CDR region (e.g. is within about 6 Å of a CDR region).

Other candidates for substitution are acceptor human framework amino acids that are unusual for a human immunoglobulin at that position. These amino acids can be substituted with amino acids from the equivalent position of the non-human donor antibody or from the equivalent positions of more typical human immunoglobulins. Other candidates for substitution are acceptor human framework amino acids that are unusual for a human immunoglobulin at that position.

An exemplary humanized antibody of the invention is a humanized form of ABC2, characterized by a mature light chain variable region of residues 23 to 128 of SEQ ID NO:2 and a mature heavy chain variable region of residues to 20 to 140 of SEQ ID NO:1 and designated HuABC2. The invention also provides variants of HuABC2. Such variants typically differ from the sequences of HuABC2 by a small number (e.g., typically no more than 1, 2, 3, 5 or 10) of replacements, deletions or insertions. Such differences are usually in the framework but can also occur in the CDRs. For example, only a subset of substitutions at positions H28, H48, H49, H68, H93 and L71 can be made. Many of the framework residues not in contact with the CDRs in the humanized mAb can accommodate substitutions of amino acids from the corresponding positions of the donor mouse mAb or other mouse or human antibodies, and even many potential CDR-contact residues are also amenable to substitution or even amino acids within the CDRs may be altered. One example of a CDR substitution is to substitute a residue in a CDR with the residue occupying the corresponding position of the human acceptor sequence used to supply variable region frameworks.

Often the replacements made in the variant humanized ABC2 sequences are conservative with respect to the replaced HuABC2 amino acids. Preferably, replacements in HuABC2 (whether or not conservative) have no substantial effect on the binding affinity or potency of the humanized mAb, that is, its ability to inhibit interactions between receptors including CD122 and IL-2 and IL-15 (e.g., the potency in some or all of the assays described in the present examples or Background of the variant humanized ABC2 antibody is essentially the same, i.e., within experimental error, as that of HuABC2). Preferably the mature variant light and heavy chain V region sequences are at least 90%, more preferably at least 95%, and most preferably at least 98% identical to the respective HuABC2 mature light and heavy chain V regions. Alternatively, other human antibody acceptor sequences, particularly those with high sequence identity to the variable region framework sequences of ABC2 are also suitable to provide the humanized antibody variable regions framework sequences.

In some variants of HuABC2, at least 1, 2, 3, 4, 5 or all 6 of the positions of acceptor to donor substitutions mentioned in connection with the exemplified antibody (i.e., H28, H48, H49, H68, H93, and L71) are preferably occupied by residues T, I, A, I, A, and Y respectively (the residues occupying the corresponding position of the mouse donor antibody heavy chain). In some variants, position H42 is occupied by a G. If the heavy chain acceptor sequence is other than the sequence encoded by DA430129 cDNA (GenBank accession number), or the light chain acceptor sequence is other than the sequence encoded by M29469 cDNA (GenBank accession number), an acceptor to donor substitution may or may not be required for the specified occupancy of a particular variable framework region position depending on whether the residue occupying the specified position is already the same between the acceptor and donor.

Another exemplary humanized antibody of the invention is a humanized form of ABC101, characterized by a mature light chain variable region of SEQ ID NO:30 and a mature heavy chain variable region of SEQ ID NO:29 and designated HuABC101. The invention also provides variants of HuABC101. Such variants typically differ from the sequences of HuABC101 by a small number (e.g., typically no more than 1, 2, 3, 5 or 10) of replacements, deletions or insertions. Such differences are usually in the framework but can also occur in the CDRs. For example, only a subset of substitutions at positions H27, H30, H48, H66, H67, H71 and L49 can be made. Many of the framework residues not in contact with the CDRs in the humanized antibody can accommodate substitutions of amino acids from the corresponding positions of the donor mouse antibody or other mouse or human antibodies, and even many potential CDR-contact residues are also amenable to substitution or even amino acids within the CDRs may be altered. One example of a CDR substitution is to substitute a residue in a CDR with the residue occupying the corresponding position of the human acceptor sequence used to supply variable region frameworks.

Often the replacements made in the variant humanized ABC101 sequences are conservative with respect to the replaced HuABC101 amino acids. Preferably, replacements in HuABC101 (whether or not conservative) have no substantial effect on the binding affinity or potency of the humanized mAb, that is, its ability to inhibit interactions between receptors including CD122 and IL-2 and IL-15 (e.g., the potency in some or all of the assays described herein of the variant humanized ABC101 antibody is essentially the same, i.e., within experimental error, as that of HuABC101). Preferably the mature variant light and heavy chain V region sequences are at least 90%, more preferably at least 95%, and most preferably at least 98% identical to the respective HuABC101 mature light and heavy chain V regions. Alternatively, other human antibody variable region framework acceptor sequences, particularly those with high sequence identity to the variable region framework sequences of ABC101 are also suitable to provide the humanized antibody framework.

In some variants of HuABC101, at least 1, 2, 3, 4, 5, 6 or all 7 of the positions of acceptor to donor substitutions mentioned in connection with the exemplified antibody (i.e., H27, H30, H48, H66, H67, H71 and L49) are preferably occupied by residues Y, T, M, Q, I, R and K respectively (the residues occupying the corresponding position of the mouse donor antibody heavy chain). If the heavy chain acceptor sequence is other than the sequence encoded by DA936142 cDNA (GenBank accession number), or the light chain acceptor sequence is other than the sequence encoded by Z46622 cDNA (GenBank accession number), an acceptor to donor substitution may or may not be required for the specified occupancy of a particular variable framework region position depending on whether the residue occupying the specified position is already the same between the acceptor and donor.

The invention can also use chimeric and veneered forms of non-human antibodies, particularly the ABC2 and ABC101 antibodies of the examples.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B-or T-cell epitopes, for example exposed residues (Padlan, Mol. Immunol. 28: 489, 1991) with residues from the corresponding positions of a human antibody sequence. The result is an antibody in which the CDRs are entirely or substantially from a non-human antibody and the variable region frameworks of the non-human antibody are made more human-like by the substitutions. Veneered forms of either the ABC2 or ABC101 antibody are included in the invention.

Human antibodies against CD122 are provided by a variety of techniques described below. Some human antibodies are selected by competitive binding experiments, by the phage display method of Winter, above, or otherwise, to have the same epitope specificity as a particular mouse antibody, such as one of the mouse monoclonals described in the examples. Human antibodies can also be screened for a particular epitope specificity by using only a fragment of CD122 as the target antigen, and/or by screening antibodies against a collection of deletion mutants of CD122.

Methods for producing human antibodies include the trioma method of Oestberg et al., Hybridoma 2: 361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. No. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991)) and phage display methods (see, e.g., Dower et al., WO 91/17271 and McCafferty et al., WO 92/01047, U.S. Pat. No. 5,877,218, 5,871,907, 5,858,657, 5,837,242, 5,733,743 and 5,565,332).

### III. Selection of Constant Region

The heavy and light chain variable regions of chimeric, humanized (including veneered), or human antibodies can be linked to at least a portion of a human constant region. The choice of constant region depends, in part, whether antibody-dependent complement and/or cellular mediated cytotoxicity is desired. For example, human isotypes IgG1 and IgG3 have complement-mediated cytotoxicity and human isotypes IgG2 and IgG4 do not. Light chain constant regions can be lambda or kappa. Antibodies can be expressed as tetramers containing two light and two heavy chains, as separate heavy chains, light chains, as Fab, Fab′, F(ab′)2, and Fv, or as single chain antibodies in which heavy and light chain variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103: 4005, 2006), or to prolong half-life in humans (see, e.g., Hinton et al., J. Biol. Chem. 279: 6213, 2004). Exemplary substitutions include a Gln at position 250 and/or a Leu at position 428 (EU numbering) for increasing the half-life of an antibody. Substitution any of positions 234, 235, 236 and/or 237 reduce affinity for Fcγ receptors, particularly FcγRI receptor (see, e.g., U.S. Pat. No. 6,624,821). An exemplary substitution is a double mutation comprising Leu-to-Ala substitutions at positions 234 and 235 (L234A and L235A, respectively) (Hazareh et al., J. Virol. 75: 12161-12168, 2001). Optionally, positions 234, 236 and/or 237 in human IgG2 are substituted with alanine and position 235 with glutamine. (See, e.g., U.S. Pat. No. 5,624,821.)

### IV. CTLA-4 Extracellular Domain

CTLA-4 is a negative regulator of immune responses. CTLA-4 specifically binds to CD80 and CD86 expressed on the surface of APCs, inhibits interaction of CD80 and CD86 with CD28 expressed on T cells, and thereby inhibits CD28-mediated co-stimulation of T cells.

An exemplary human form of CTLA-4 has been assigned Swiss Prot P16410. This form has 223 amino acids of which residues 1-35 are a signal peptide, residues 36-161 are an extracellular domain, residues 162-182 are a transmembrane domain and residues 173-223 are a cytoplasmic domain. An exemplary extracellular domain is assigned SEQ ID NO:10 herein, which corresponds to residues 38 to 160 of the Swiss Prot sequence. Reference to the extracellular domain of CTLA-4 includes SEQ ID NO:10 and variants having up to 1, 2, 3, 4, 5, 6, 7, 8, 9 or 10 substitutions, deletions or insertions, and/variants having at least 90, 95, 95 or 99% identity with SEQ ID NO: 10. Such variations can be natural or induced, examples of which are provided in the annotation of P16410. Variations can also represent alternative amino acids present in CTLA-4 of other species. CTLA-4 has been subject to systematic mutagenesis to identify substitutions enhancing or inhibiting binding to CD80 and CD86 (Xu et al., J. Immunol. 189: 4470-4477, 2012). Examples of high-affinity mutations includes K28H, K28T, A29H, A29T, A29Y, A29W, and T30G. These and other mutations are named by the wild-type amino acid residue on the left, their position in SEQ ID NO:10 in the middle, and an amino acid residue after mutagenesis on the right. Mutations at T at position 51, M at position 53, M at position 54, L at position 58, and L at position 61 also favorably impact binding to one or both ligands. The motif 97MYPPPY102 was intolerant to mutations, especially for interaction with CD80, whereas beneficial mutations that alter the affinity balance for CD80 and CD86 clustered adjacent to this conserved motif (K at position 93, L at position 96, and L at position 104). CTLA-4 mutants with stronger binding to CD80 and CD86 are also reported in Douthwaite et al. (J. Immunol. 198: 528-537, 2017) and Oshima et al. (Protein Eng. Des. Sel. 29: 159-167, 2016). Preferred variants that enhance binding include A29Y and L104E. These and other mutations are numbered by their position in SEQ ID NO:10 or if present in a sequence with a different number of residues than SEQ ID NO: 10, the corresponding position of SEQ ID NO:10 to the position of the mutation in the other sequence when the respective sequences are maximally aligned. Additional flanking amino acids from P16410 may or may not be included at one or both sides of SEQ ID NO:10.

CTLA-4 and its variants show specific binding to CD80 and CD86. An exemplary form of human CD80 also known as B7-1 or BB1 is provided by Swiss-Prot P33681. An exemplary form of human CD86 also known as B7-2 is provided by Swiss-Prot P42081. CD80 and CD86 in turn interact with CD28. An exemplary form of human CD28 is provided by Swiss-Prot P10747.

Belatacept is a recombinant protein comprising the extracellular domain of human CTLA-4 (cytotoxic T-lymphocyte-associated protein 4; also called CD152) fused to the Fc region of human gamma-1 heavy chain (CTLA4-Ig) (SEQ ID NO:49). The CTLA-4 region of belatacept carries two amino acid substitutions, one from an alanine residue to a tyrosine residue at position 29 (A29Y) and another from a leucine residue to a glutamate residue at position 104 (L104E), to enhance the binding to CD80 (also called B7-1) and CD86 (also called B7-2) (Larsen et al., Am. J. Transplant. 5: 443-453, 2005; Oshima et al., Protein Eng. Des. Sel. 29: 159-167, 2016). Belatacept was approved for marketing in the U.S. for prevention of renal allograft rejection. For reviews of belatacept, see Perez et al., Transplantation 102: 1440-1452, 2008; Gallo et al., PLOS ONE 15: e0240335, 2020.

Abatacept, which is a predecessor of belatacept, is also a recombinant protein comprising a wildtype extracellular domain of human CTLA-4 fused to the Fc region of human gamma-1 heavy chain. No amino acid substitutions are introduced in the CTLA-4 region. Abatacept, like belatacept, works as an immune suppresser by blocking the interaction of CD80 and CD86 with CD28. Abatacept was approved for marketing in the U.S. for treatment of adult rheumatoid arthritis, polyarticular juvenile idiopathic arthritis, and adult psoriatic arthritis. For reviews of abatacept, see Moreland et al., Nat. Rev. Drug Disc. 5: 185-186, 2006; Esensten et al., Immunity 44: 973-988, 2016. Abatacept has been reported effective for inhibiting renal allograft rejection (Badell et al., Am. J. Transplant. 19: 2342-2349, 2019). Binding of a CTLA-4 extracellular domain moiety, such as abatacept or belatacept to CD80 and CD86 inhibits immune responses by inhibiting CD28-mediated Signal 2 of the T cell activation pathway.

### V. Bifunctional Molecules

Bifunctional molecules of the invention combine an antibody against CD122 as further described above with an extracellular domain of CTLA-4 described above. The extracellular domain of CTLA-4 can be linked to a C-terminus of a heavy chain of the antibody as a fusion protein. Thus, an exemplary bifunctional molecule comprises an antibody heavy chain comprising a mature heavy chain variable region and a heavy chain constant region fused at the C-terminus of the heavy chain constant region to a CTLA-4 extracellular domain plus a light chain comprising a mature light chain variable region and a light chain constant region. An extracellular domain of CTLA-4 can also be linked to a C-terminus of a light chain. Thus, another exemplary bifunctional molecule comprises an antibody heavy chain comprising a mature heavy chain variable region and a heavy chain constant region plus a light chain comprising a mature light chain variable region and a light chain constant region fused at the C-terminus of the light chain constant region to a CTLA-4 extracellular domain. The extracellular domain of CTLA-4 can also be linked to the N-terminus of the heavy or light chain variable region of an antibody. Thus, another exemplary bifunctional molecule comprises an antibody comprising a mature heavy chain variable region and a heavy chain constant region plus a light chain comprising a mature light chain variable region and a light chain constant region linked at the N-terminus of the heavy chain variable region or light chain variable region to the extracellular domain of CTLA-4. Linkage of the heavy or light chain to the CTLA-4 can be direct or indirect, such as via a flexible peptide linker. Flexible peptide linkers can be formed predominantly or exclusively from glycine and serine residues, such as SEQ ID NOS:11, 36 and 50. The heavy and light chains can associate with one another as in a normal antibody. Also two pairs of associated heavy and light chains can dimerize with one another to form a heterodimeric structure analogous to a normal antibody except with a CTLA-4 extracellular domain linked to the N-or C-terminus of the heavy or light chains. The heavy chain constant region can include any or all of the regions present in a normal heavy chain constant region, such as CH1, hinge, CH2 and CH3 optionally with linkage to the extracellular domain of CTLA-4 being through the C-terminus. When the extracellular domain of CTLA-4 is fused to the C-terminus of an antibody heavy chain constant region, the fusion of the CTLA-4 extracellular domain to the C-terminus of a heavy chain constant region is the opposite to that in abatacept or belatacept, where the CTLA-4 extracellular domain is fused to the N-terminus of a heavy chain constant region.

In an exemplary bifunctional molecule based on the HuABC2 antibody, the mature heavy chain is linked to the CTLA-4 extracellular domain as a chain comprising SEQ ID NO:13 or 16 and the mature light chain comprises SEQ ID NO:9. In another such exemplary bifunctional molecule, the mature light chain is linked to the CTLA-4 extracellular domain as a chain comprising SEQ ID NO:34 and the mature heavy chain comprises SEQ ID NO:8 or 33 provided the C-terminal lysine can be omitted. In another such exemplary bifunctional molecule, the mature heavy chain is linked to the CTLA-4 extracellular domain as a chain comprising SEQ ID NO:39 provided the C-terminal lysine can be omitted, and the mature light chain comprises SEQ ID NO:9. In another such exemplary bifunctional molecule, the mature light chain is linked to the CTLA-4 extracellular domain as a chain comprising SEQ ID NO:42, and the mature heavy chain comprises SEQ ID NO: 8 or 33 provided the C-terminal lysine can be omitted.

In an exemplary bifunctional molecule based on the HuABC101 antibody, the mature heavy chain is linked to the CTLA-4 extracellular domain as a chain comprising SEQ ID NO:57 and the mature light chain comprises SEQ ID NO:54. In another such exemplary bifunctional molecule, the mature heavy chain is linked to the CLTA-4 extracellular domain as a chain comprising SEQ ID NO:56 provided the C-terminal lysine can be omitted, and the mature light chain comprises SEQ ID NO:54.

In an exemplary bifunctional molecule based on the HuABC77 antibody, the mature heavy chain is linked to the CTLA-4 extracellular domain as a chain comprising SEQ ID NO:71 and the mature light chain comprises SEQ ID NO:67. In another such bifunctional molecule, the mature heavy chain is linked to the CTLA-4 extracellular domain as a chain comprising SEQ ID NO:70 provided the C-terminal lysine can be omitted, and the mature light chain comprises SEQ ID NO:67.

Such bifunctional molecules show specific binding to each of CD122, CD80 and CD86, inhibiting CD122 from interacting with ligands IL-2 and IL-15 and inhibiting CD80 and CD86 from interacting with CD28. Optionally, the binding affinity of a bifunctional molecule for CD122 is the same within experimental error or within a factor of 5, 3, or 2 of that of the anti-CD122 antibody incorporated into a bifunctional molecule. Optionally, the inhibition of CD122 binding to IL-2 or IL-15 by a bifunctional molecule is the same within experimental error or within a factor of 5, 3, or 2 of that of the anti-CD122 antibody incorporated into the bifunctional molecule alone. Optionally, the binding affinity of a bifunctional molecule for CD80 and CD86 is the same within experimental error or within a factor of 5, 3 or 2 of that of an extracellular domain of CTLA-4 incorporated into the bifunctional molecule linked to a heavy chain constant region (as for, example, in abatacept or belatacept). Optionally, the inhibition of CD80 and/or CD86 binding to CD28 by the bifunctional molecule is the same within experimental error or within a factor of 5, 3 or 2 of that of the extracellular domain of CTLA-4 linked to a constant or variable region incorporated into the bifunctional molecule. Optionally, a bifunctional molecule shows greater inhibition of immune response or greater efficacy in treating an immune disorder than either its antibody or extracellular domain components can achieve individually for equimolar doses. Optionally, a bifunctional molecule shows at least additive and preferably a synergistic increase in inhibition or efficacy compared with its antibody or extracellular domain components individually. Optionally a bifunctional molecule shows greater inhibition or efficacy compared with co-administration of its component antibody and extracellular domain in equimolar ratio as separate molecules comparing an equal number of moles of each agent. In comparisons involving an extracellular domain not part of a bifunctional molecule, the extracellular domain can be used alone or fused to an IgG constant region at either its N or C-terminal. Optionally, a bifunctional molecule shows greater inhibition or efficacy compared with co-administration of its component anti-CD122 antibody and either abatacept or belatacept, the comparison being performed with an equimolar amount of each agent.

### VI. Expression of Bifunctional Molecules

Bifunctional molecules of the invention are typically produced by recombinant expression using techniques analogous to expression of antibodies. Nucleic acids encoding the heavy chain linked to a CTLA-4 extracellular domain and a light chain or nucleic acids encoding a heavy chain and the light chain linked to a CTLA-4 extracellular domain are expressed to produce the encoded polypeptides, which associate to form a bifunctional molecule. Recombinant nucleic acid constructs typically include an expression control sequence operably linked to coding sequences of these chains, chains, including naturally associated or heterologous promoter regions. Preferably, the expression control sequences are eukaryotic promoter systems in a vector or vectors capable of transforming or transfecting eukaryotic host cells. Once the vector or vectors has been incorporated into the appropriate host, the host is maintained under conditions suitable for high level expression of the nucleotide sequences, and the collection and purification of the bifunctional molecules.

Mammalian cells are a preferred host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines, various COS cell lines, Hela cells, HEK293 cells, L cells, and non-antibody-producing myelomas including Sp2/0 and NSO. Preferably, the cells are nonhuman. Expression vectors for these cells can include expression control sequences, such as an origin of replication, a promoter, an enhancer (Queen et al., Immunol. Rev. 89: 49 (1986)), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., J. Immunol. 148: 1149 (1992).

Once expressed, bifunctional molecules can be purified according to standard procedures of the art, including HPLC purification, column chromatography, gel electrophoresis and the like (see generally, Scopes, Protein Purification (Springer-Verlag, NY, 1982)).

### VII. Pharmaceutical Compositions and Methods of Treatment

Bifunctional molecules can be used for suppressing various undesirable immune response particularly those mediated by any of CD122, CD80 and CD86, and their ligands or receptors, interaction of which is inhibited by the present bifunctional molecules.

One category of immune disorders treatable by bifunctional molecules of the invention is transplant rejection. When allogeneic cells or organs (e.g., skin, kidney, liver, heart, lung, pancreas and bone marrow) are transplanted into a host (i.e., the donor and donee are different individual from the same species), the host immune system is likely to mount an immune response to foreign antigens in the transplant (host-versus-graft disease) leading to destruction of the transplanted tissue. The bifunctional molecules of the present invention are useful, inter alia, to block alloantigen-induced immune responses in the donee.

A related use for bifunctional molecules of the present invention is in modulating the immune response involved in “graft versus host” disease (GVHD). GVHD is a potentially fatal disease that occurs when immunologically competent cells are transferred to an allogeneic recipient. In this situation, the donor's immunocompetent cells may attack tissues in the recipient. Tissues of the skin, gut epithelia and liver are frequent targets and may be destroyed during the course of GVHD. The disease presents an especially severe problem when immune tissue is being transplanted, such as in bone marrow transplantation; but less severe GVHD has also been reported in other cases as well, including heart and liver transplants.

A further situation in which immune suppression is desirable is in treatment of autoimmune diseases such as type 1 diabetes, Crohn's disease, ulcerative colitis, multiple sclerosis, stiff person syndrome, rheumatoid arthritis, myasthenia gravis, lupus erythematosus, celiac disease, psoriasis, uveitis, alopecia areata, and primary biliary cirrhosis. In these autoimmune diseases, the body develops a cellular and/or humoral immune response against one of its own antigens leading to destruction of that antigen, and potentially crippling and/or fatal consequences. Autoimmune diseases are treated by administering a bifunctional molecule of the invention.

Other autoimmune diseases treatable by the bifunctional molecules of the invention include asthma, allergies, vitiligo, polymyositis, allergic dermatitis, and Behçet's disease. Other autoimmune diseases which can be treated include idiopathic thrombocytopeniarpura, Kawasaki disease, Guillain-Barre syndrome, chronic inflammatory demyelinating systemic lupus erythematosus, autoimmune neutropenia type 1 diabetes, acute disseminated encephalomyelitis, acute motor axonal neuropathy, Addison's disease, adiposis dolorosa, adult-onset Still's disease, ankylosing spondylitis, anti-glomerular basement membrane nephritis, anti-neutrophil cytoplasmic antibody-associated vasculitis, anti-N-methyl-D-aspartate receptor encephalitis, antiphospholipid syndrome, antisynthetase syndrome, aplastic anemia, autoimmune angioedema, autoimmune encephalitis, autoimmune enteropathy, autoimmune hemolytic anemia, autoimmune hepatitis, autoimmune inner ear disease, autoimmune lymphoproliferative syndrome, autoimmune oophoritis, autoimmune orchitis, autoimmune pancreatitis, autoimmune polyendocrine syndrome, autoimmune polyendocrine syndrome type 2, autoimmune polyendocrine syndrome type 3, autoimmune progesterone dermatitis, autoimmune retinopathy, autoimmune thrombocytopenia purpura, autoimmune thyroiditis, autoimmune urticaria, autoimmune uveitis, Balo concentric sclerosis, Behçet's disease, Bickerstaff's encephalitis, bullous pemphigoid, chronic fatigue syndrome, Churg-Strauss syndrome, cicatricial pemphigoid, Cogan syndrome, cold agglutinin disease, complex regional pain syndrome, CREST syndrome, dermatitis herpetiformis, dermatomyositis, discoid lupus erythematosus, endometriosis, enthesitis, enthesitis-related arthritis, eosinophilic esophagitis, eosinophilic fasciitis, epidermolysis bullosa acquisita, erythema nodosum, essential mixed cryoglobulinemia, Evans syndrome, Felty syndrome, fibromyalgia, gastritis, gestational pemphigoid, giant cell arteritis, Goodpasture syndrome, Graves' disease, Graves ophthalmopathy, Hashimoto's encephalopathy, Hashimoto thyroiditis, Henoch-Schonlein purpura, hidradenitis suppurativa, idiopathic dilated cardiomyopathy, idiopathic inflammatory demyelinating diseases, IgA nephropathy, IgG4-related systemic disease, inclusion body myositis, inflammatory bowel disease (IBD), intermediate uveitis, interstitial cystitis, juvenile arthritis, Kawasaki's disease, Lambert-Eaton myasthenic syndrome, leukocytoclastic vasculitis, lichen planus, lichen sclerosus, ligneous conjunctivitis, linear IgA disease, lupus nephritis, lupus vasculitis, Lyme disease, Ménière's disease, microscopic colitis, microscopic polyangiitis, mixed connective tissue disease, Mooren's ulcer, morphea, Mucha-Habermann disease, myocarditis, myositis, neuromyelitis optica, neuromyotonia, opsoclonus myoclonus syndrome, optic neuritis, Ord's thyroiditis, palindromic rheumatism, paraneoplastic cerebellar degeneration, Parry Romberg syndrome, Parsonage-Turner syndrome, pediatric autoimmune neuropsychiatric disorder associated with Streptococcus, pemphigus vulgaris, pernicious anemia, pityriasis lichenoides et varioliformis acuta, POEMS syndrome, polyarteritis nodosa, polymyalgia rheumatica, polymyositis, postmyocardial infarction syndrome, postpericardiotomy syndrome, primary biliary cirrhosis, primary immunodeficiency, primary sclerosing cholangitis, progressive inflammatory neuropathy, psoriatic arthritis, pure red cell aplasia, pyoderma gangrenosum, Raynaud's phenomenon, reactive arthritis, relapsing polychondritis, restless leg syndrome, retroperitoneal fibrosis, rheumatic fever, rheumatoid vasculitis, sarcoidosis, Schnitzler syndrome, scleroderma, Sjogren's syndrome, subacute bacterial endocarditis, Susac's syndrome, Sydenham chorea, sympathetic ophthalmia, systemic scleroderma, thrombocytopenia, Tolosa-Hunt syndrome, transverse myelitis, undifferentiated connective tissue disease, urticaria, urticarial vasculitis, and vasculitis.

Although immune disorders can be subdivided into categories as indicated above, the categories are not necessarily mutually exclusive, nor is treatment dependent on the category in which a disorder is placed.

Other disorders treatable by bifunctional molecules of the invention include cancers, particularly hematological malignancies, such as leukemia (e.g., T cell large granular lymphocyte leukemia) or lymphoma. Some such cancers show detectable levels of CD122, CD80 and/or CD86 measured at either the protein (e.g., by immunoassay using one of the exemplified bifunctional molecules) or mRNA level. Some such cancers show elevated levels of CD122, CD80 and/or CD86 relative to noncancerous tissue of the same type, preferably from the same subject. Optionally, a level of CD122, CD80 or CD86 in a cancer is measured before performing treatment.

Bifunctional molecules are administered in an effective regime meaning a dosage, route of administration and frequency of administration that delays the onset, reduces the severity, inhibits further deterioration, and/or ameliorates at least one sign or symptom of a disorder. If a subject is already suffering from a disorder, the regime can be referred to as a therapeutically effective regime. If a subject is at elevated risk of the disorder relative to the general population but is not yet experiencing symptoms, the regime can be referred to as a prophylactically effective regime. In some instances, therapeutic or prophylactic efficacy can be observed in an individual subject relative to historical controls or past experience in the same subject. In other instances, therapeutic or prophylactic efficacy can be demonstrated in a preclinical or clinical trial in a population of treated subjects relative to a control population of untreated subjects.

Exemplary dosage for a bifunctional molecule is 0.1-20 mg/kg, or 0.5-5 mg/kg body weight (e.g., 0.5, 1, 2, 3, 4 or 5 mg/kg) or 10-1500 mg as a fixed dosage. The dosage depends on the condition of the subject and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Administration into the systemic circulation by intravenous or subcutaneous administration is preferred. Intravenous administration can be, for example, by infusion over a period such as 30-90 min.

The frequency of administration depends on the half-life of the bifunctional molecule in the circulation, the condition of the subject and the route of administration among other factors. The frequency can be daily, weekly, monthly, quarterly, or at irregular intervals in response to changes in the subject's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of a chronic disorders between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, a bifunctional molecule can be administered at regular intervals, e.g., weekly, fortnightly, monthly, quarterly, every six months for at least 1, 5 or 10 years, or the life of the subject.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more pharmacologically acceptable carriers, diluents, excipients or auxiliaries. The formulation depends on the route of administration chosen. For injection, bifunctional molecules can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, bifunctional molecules can be in lyophilized form for constitution with a suitable vehicle, e.g., sterile pyrogen-free water, before use.

Treatment with bifunctional molecules of the invention can be combined with other treatments effective against the disorder being treated. For treatment of immune disorders, conventional treatments include mast cell degranulation inhibitors, corticosteroids, nonsteroidal anti-inflammatory drugs, and stronger anti-inflammatory drugs such as azathioprine, cyclophosphamide, leukeran, FK506 and cyclosporine. Biologic anti-inflammatory agents, such as Tysabri® (natalizumab) or Humira® (adalimumab), can also be used. When used in treating cancer, the bifunctional molecules of the invention can be combined with chemotherapy, radiation, stem cell treatment, surgery or treatment with other biologics such as Herceptin® (trastuzumab) against the HER2 antigen, Avastin® (bevacizumab) against VEGF, or antibodies to the EGF receptor, such as (Erbitux®, cetuximab), and Vectibix® (panitumumab). Chemotherapy agents include chlorambucil, cyclophosphamide or melphalan, carboplatinum, daunorubicin, doxorubicin, idarubicin, and mitoxantrone, methotrexate, fludarabine, and cytarabine, etoposide or topotecan, vincristine and vinblastine.

### VIII. Other Applications

The bifunctional molecules can be used for detecting CD122, CD80 and/or CD86 in the context of clinical diagnosis or treatment or in research. For example, the bifunctional molecules can be used to detect presence of CD122, CD80 and/or CD86 on T-cells as an indication a subject is suffering from an immune mediated disorder amenable to treatment. Expression of CD122, CD80 and/or CD86 on a cancer also provides an indication that the cancer is amenable to treatment with the bifunctional molecules of the present invention. The bifunctional molecules can also be sold as research reagents for laboratory research in detecting T-cells and their response to various stimuli. In such uses, bifunctional molecules can be labeled with fluorescent molecules, spin-labeled molecules, enzymes or radioisotopes, and can be provided in the form of kit with all the necessary reagents to perform the assay for CD122, CD80 or CD86. The bifunctional molecules can also be used to purify CD122, CD80 and/or CD86, e.g., by affinity chromatography.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLE 1

### General Methods and Materials

Gene cloning, mutagenesis, plasmid construction, ELISA, and flow cytometry were carried out following standard laboratory techniques such as those described by Green and Sambrook (Molecular Cloning, A Laboratory Manual, 4th ed., 2012, Cold Spring Harbor Laboratory Press, Cold Spring Harbor, NY), Greenfield (Antibodies, A Laboratory Manual, 2nd ed., 2014, Cold Spring Harbor Laboratory Press, Cold Spring Harbor, NY), Kostelny et al. (Int. J. Cancer 93: 556-565, 2001), Cole et al. (J. Immunol. 159: 3613-3621, 1997) and Tsurushita et al. (Methods 36: 69-83, 2005), and in vendors' protocols.

HuABC2 is a humanized anti-CD122 IgG1/kappa monoclonal antibody that (i) binds to the beta chain subunit of IL-2 and IL-15 receptors (also called CD122) and (ii) blocks the interaction of IL-2 and IL-15 with their respective receptors (U.S. Pat. No. 9,028,830). Amino acid sequence of the heavy chain variable region (VH) of HuABC2 is

(SEQ ID NO: 1)
MKLWLNWVFLLTLLHGIQCEVQLVESGGGLVQPGGSLRLSCAASGFTFSD
FYMEWVRQAPGKGLEWIAASRNKANDYTTEYSASVKGRFIVSRDDSKNSL
YLQMNSLKTEDTAVYYCARSYYRYDGMDYWGQGTTVTVSS.

The mature HuABC2 VH starts at a glutamate residue of position 20 in SEQ ID NO:1.

Amino acid sequence of the mature light chain variable region (VL) of HuABC2 is

(SEQ ID NO: 2)
MDFQVQIFSFLLISASVIVSRGEIVLTQSPATLSLSPGERATLSCSAISS
VSYMYWYQQKPGQAPRLLIYDTSNLVSGVPARFSGSGSGTDYTLTISSLE
PEDFAVYYCQQWNTYPYTFGGGTKVEIK.

The mature HuABC2 VL starts at a glutamate residue of position 23 in SEQ ID NO:2.

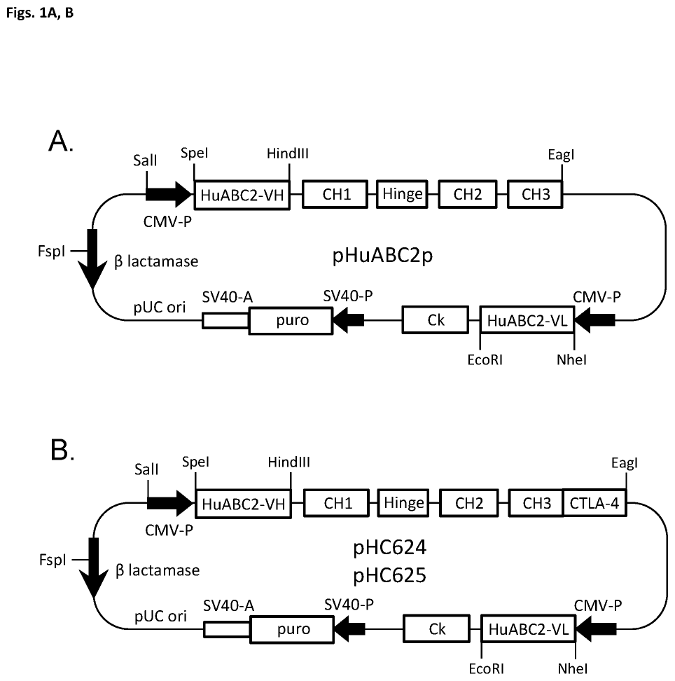

The schematic structure of the plasmid vector pHuABC2p for production of HuABC2 in mammalian cells is shown in FIG. 1A. Proceeding clockwise from the Sall site in the figure, pHuABC2p contains the heavy chain transcription unit starting with the human cytomegalovirus (CMV) major immediate early promoter and enhancer (CMV-P) to initiate transcription of the antibody heavy chain gene. The CMV promoter is followed by the VH exon encoding HuABC2 VH, a genomic sequence containing the human gamma-1 heavy chain constant region including the CH1, Hinge, CH2 and CH3 exons with the intervening introns, and the polyadenylation site following the CH3 exon. After the heavy chain gene sequence, the light chain transcription unit begins with the CMV promoter, followed by the exon encoding HuABC2 VL and a genomic sequence containing the human kappa chain constant region (Ck) exon with part of the intron preceding it, and the polyadenylation site following the Ck exon. The light chain gene is then followed by the SV40 early promoter (SV40-P), the puromycin N-acetyl-transferase gene (puro) for resistance to puromycin, and a segment containing the SV40 polyadenylation site (SV40-A). Finally, the plasmid contains a part of the plasmid pUC19, comprising the bacterial origin of replication (pUC ori) and beta-lactamase gene (β lactamase). Locations of restriction enzyme sites are shown in the figure. Arrows indicate orientation of transcription.

Amino acid sequences of the CH1, hinge, CH2, and CH3 regions of human gamma-1 heavy chain encoded in pHuABC2p are

(SEQ ID NO: 3)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV,
(SEQ ID NO: 4)
EPKSCDKTHTCPPCP,
(SEQ ID NO: 5)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK,
and
(SEQ ID NO: 6)
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK,

respectively. Amino acid sequence of the Ck region encoded in pHuABC2p is

(SEQ ID NO: 7)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC.

Amino acid sequence of mature heavy chain encoded in pHuABC2p is

(SEQ ID NO: 8)
EVQLVESGGGLVQPGGSLRLSCAASGFTFSDFYMEWVRQAPGKGLEWIAA
SRNKANDYTTEYSASVKGRFIVSRDDSKNSLYLQMNSLKTEDTAVYYCAR
SYYRYDGMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K.

Amino acid sequence of mature light chain encoded in pHuABC2p is

(SEQ ID NO: 9)
EIVLTQSPATLSLSPGERATLSCSAISSVSYMYWYQQKPGQAPRLLIYDT
SNLVSGVPARFSGSGSGTDYTLTISSLEPEDFAVYYCQQWNTYPYTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

CHO-K1 cells stably transfected with pHuABC2p (CHO-K1/pHuABC2p) for expression of HuABC2 were grown in SFM4CHO media (HyClone Laboratories, Logan, UT). For purification of HuABC2, culture supernatants of CHO-K1/pHuABC2p cells were loaded onto a protein-A Sepharose® column (HiTrap® MabSelect SuRe™, GE Healthcare, Piscataway, NJ). The column was washed with PBS before HuABC2 was eluted with 0.1 M glycine-HCl buffer (pH 3.0) containing 0.1 M NaCl. After neutralization with 1 M Tris-HCl (pH 8.0), the buffer of eluted antibody was changed to PBS by dialysis. Antibody concentration was determined by measuring absorbance at 280 nm (1.4 OD=1 mg/ml).

### EXAMPLE 2

### Bifunctional Molecules Binding to CD80, CD86 and CD122

Binding of CD80 or CD86 on antigen-presenting cells to CD28 on T cells leads to enhancement of the immune responses. CTLA-4 binds to CD80 and CD86 and blocks their interaction with CD28, which results in suppression of T cell activation (Esensten et al., Immunity 44: 973-988, 2016).

To equip HuABC2 with the ability to block the function of an immune costimulatory molecule CD28, the CH3 exon in pHuABC2p was modified in such a way to encode the extracellular domain of human CTLA-4 (SEQ ID NO:10) fused at its N-terminal to the penultimate amino acid residue (Gly) of CH3 (CH3-CTLA4 fusion). A flexible polypeptide linker (SGGGGSG; SEQ ID NO:11) was placed between CH3 and CTLA-4 in CH3-CTLA4 fusion. Amino acid sequence of CH3-CTLA4 fusion is

(SEQ ID NO: 12)
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGSGGGGSGMHVAQPAVVLASSRGIASFVCEYASPGKATEVRVTVL
RQADSQVTEVCAATYMMGNELTFLDDSICTGTSSGNQVNLTIQGLRAMDT
GLYICKVELMYPPPYYLGIGNGTQIYVIDPEPCPDSD.

In addition, leucine residues at positions 234 and 235 (EU numbering) in CH2 were both changed to alanine residues to eliminate binding to Fc gamma receptors (Hezareh et al., J. Virol. 75: 12161-12168, 2001). The resulting expression vector derived from pHuABC2p was termed pHC624. The schematic structure of pHC624 is shown in FIG. 1B.

Amino acid sequence of the mature heavy chain of HuABC2 fused at the C-terminus to the linker (underlined) followed by the extracellular domain of CTLA-4 encoded in pHC624 is

(SEQ ID NO: 13)
EVQLVESGGGLVQPGGSLRLSCAASGFTFSDFYMEWVRQAPGKGLEWIAA
SRNKANDYTTEYSASVKGRFIVSRDDSKNSLYLQMNSLKTEDTAVYYCAR
SYYRYDGMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
SGGGGSGMHVAQPAVVLASSRGIASFVCEYASPGKATEVRVTVLRQADSQ
VTEVCAATYMMGNELTFLDDSICTGTSSGNQVNLTIQGLRAMDTGLYICK
VELMYPPPYYLGIGNGTQIYVIDPEPCPDSD.

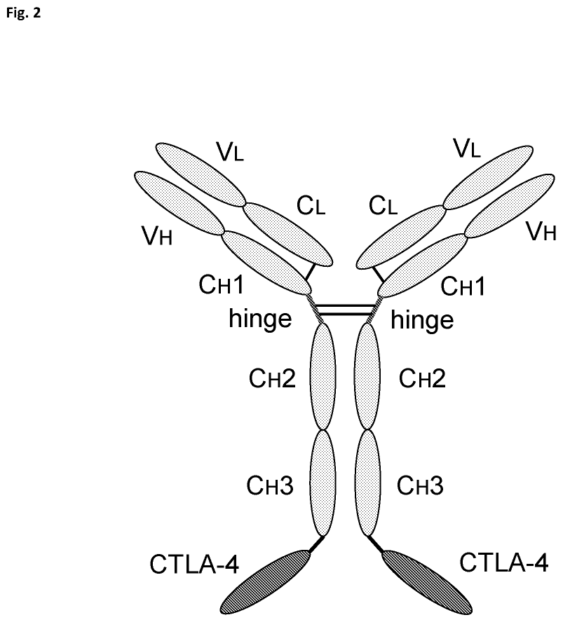

Amino acid sequence of mature light chain encoded in pHC624 is same as the mature light chain sequence encoded in pHuABC2p (SEQ ID NO:9). Modified HuABC2 fused to CTLA-4 at the end of the heavy chain, which is encoded by pHC624, was termed HuABC2-CTLA4. The schematic structure of HuABC2-CTLA4 is shown in FIG. 2.

The extracellular domain of CTLA-4 (SEQ ID NO:10) was modified by substitution of two amino acid residues. One of them is a substitution of an alanine residue to a tyrosine reside at position 29 (A29Y) of SEQ ID NO:10. The other is a substitution of a leucine residue to a glutamate residue at position 104 (L104E) of SEQ ID NO:10. Amino acid sequence of the extracellular domain of the resulting modified CTLA-4 (variant CTLA-4) is

(SEQ ID NO: 14)
MHVAQPAVVLASSRGIASFVCEYASPGKYTEVRVTVLRQADSQVTEVCAA
TYMMGNELTFLDDSICTGTSSGNQVNLTIQGLRAMDTGLYICKVELMYPP
PYYEGIGNGTQIYVIDPEPCPDSD.

The DNA fragment encoding the variant CTLA-4 was used to replace the region encoding the wild-type CTLA-4 in pHC624.

The resulting expression vector was named pHC625 (also called pHC622). The schematic structure of pHC625 is shown in FIG. 1B.

Amino acid sequence of the modified CH3-CTLA4 fusion encoded in pHC625 and pHC622 is

(SEQ ID NO: 15)
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGSGGGGSGMHVAQPAVVLASSRGIASFVCEYASPGKYTEVRVTVL
RQADSQVTEVCAATYMMGNELTFLDDSICTGTSSGNQVNLTIQGLRAMDT
GLYICKVELMYPPPYYEGIGNGTQIYVIDPEPCPDSD.

The linker sequence is underlined.

Amino acid sequence of the mature heavy chain of HuABC2 fused at the C-terminus to the linker (underlined) followed by the extracellular domain of the variant CTLA-4 encoded in pHC625 and pHC622 is

(SEQ ID NO: 16)
EVQLVESGGGLVQPGGSLRLSCAASGFTFSDFYMEWVRQAPGKGLEWIAA
SRNKANDYTTEYSASVKGRFIVSRDDSKNSLYLQMNSLKTEDTAVYYCAR
SYYRYDGMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
SGGGGSGMHVAQPAVVLASSRGIASFVCEYASPGKYTEVRVTVLRQADSQ
VTEVCAATYMMGNELTFLDDSICTGTSSGNQVNLTIQGLRAMDTGLYICK
VELMYPPPYYEGIGNGTQIYVIDPEPCPDSD.

Amino acid sequence of mature light chain encoded in pHC625 and pHC622 is same as the mature light chain sequence encoded in pHuABC2p (SEQ ID NO:9). Modified HuABC2 fused to the variant CTLA-4 at the end of the heavy chain, which is encoded in pHC625 and pHC622, was termed HuABC2-CTLA4YE.

### EXAMPLE 3

### Transient Expression and Characterization of Bifunctional Molecules Binding to CD80, CD86 and CD122

The expression vectors pHC624 and pHC625 were individually transfected into the human embryonic kidney cell line HEK293 by the procedure of Durocher et al. (Nucl. Acids Res. 30:e9 2002) for secretion of HuABC2-CTLA4 and HuABC2-CTLA4YE, respectively, in culture supernatants. HEK293 cells were grown in DME media containing 10% fetal bovine serum (FBS; HyClone, Logan, UT) at 37° C. in a 7.5% CO2 incubator. Expression level of HuABC2-CTLA4 and HuABC2-CTLA4YE in culture supernatants was measured by sandwich ELISA. In a typical experiment, wells of an ELISA plate were coated with goat anti-human gamma heavy chain polyclonal antibody in PBS, washed with Wash Buffer (PBS containing 0.05% Tween 20), and blocked with Blocking Buffer (PBS containing 2% skim milk and 0.05% Tween 20). After washing the wells with Wash Buffer, test samples appropriately diluted in ELISA Buffer (PBS containing 1% skim milk and 0.025% Tween 20) were applied to the wells. Purified HuABC2 appropriately diluted in ELISA Buffer was used as a standard. After incubation for 1 hr and washing with Wash Buffer, bound antibodies were detected using HRP-conjugated goat anti-human kappa chain polyclonal antibody. After incubation for 30 min and washing with Wash Buffer, color development was initiated by adding ABTS substrate (Sigma-Aldrich, St. Louis, MO) and stopped with 2% oxalic acid. Absorbance was read at 405 nm.

Binding of HuABC2-CTLA4 and HuABC2-CTLA4YE to human CD122, CD80 and CD86 was analyzed by ELISA. Wells of an ELISA plate were coated with 1 μg/ml of either CD122 (Human IL-2R beta/CD 122 Protein, Fc Tag; Cat. No. ILB-H5253, Acro Biosystems, Newark, DE), CD80 (Human B7-1/CD80 Protein, Mouse IgG2a Tag; Cat. No. B71-H52A4, Acro Biosystems) or CD86 (Human B7-2/CD86 Protein, Fc Tag; Cat. No. CD6-H5257, Acro Biosystems) in PBS. After blocking with Blocking Buffer and washing the wells with Wash Buffer, transiently expressed HuABC2-CTLA4 and HuABC2-CTLA4YE at 0.5 μg/ml and serial 2-fold dilutions in ELISA Buffer were applied to the wells in duplicates. Purified HuABC2 at 0.5 μg/ml and serial 2-fold dilutions was used as a control. After incubation for 1 hr at room temperature and washing with Wash Buffer, bound antibodies were detected using HRP-conjugated goat anti-human kappa chain polyclonal antibody. After incubation for 30 min and washing with Wash Buffer, color development was initiated by adding ABTS substrate and stopped with 2% oxalic acid. Absorbance was read at 405 nm.

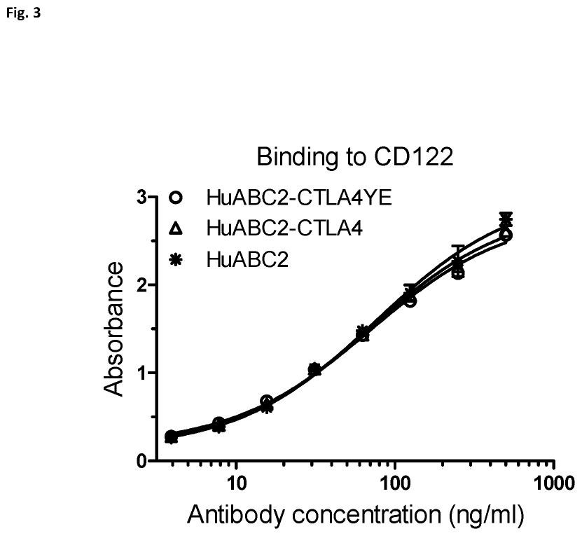

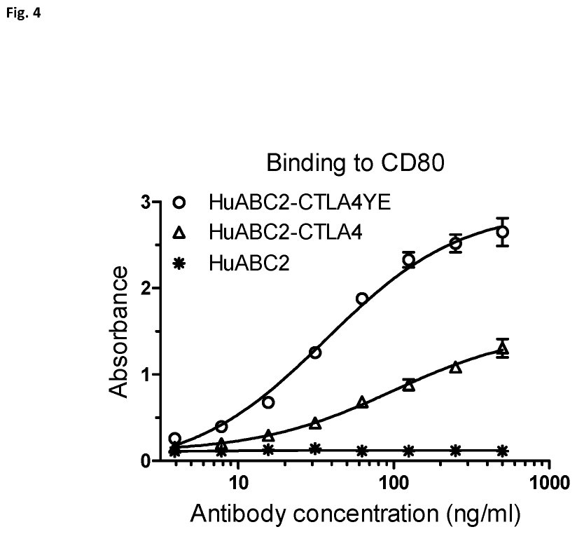

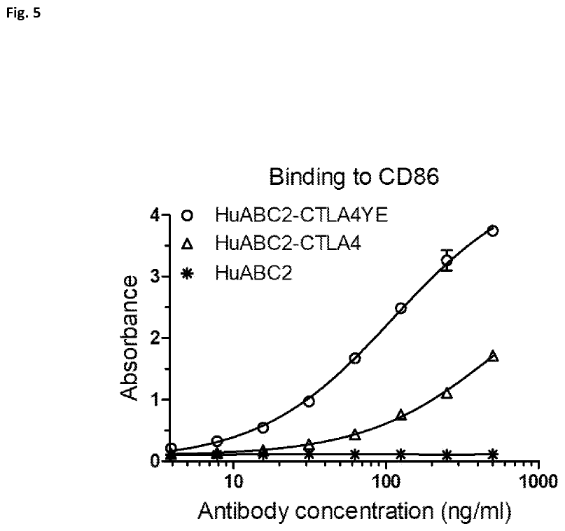

FIGS. 3, 4 and 5 show the results of the ELISA experiments for binding of HuABC2, HuABC2-CTLA4 and HuABC2-CTLA4YE to each of human CD122, CD80 and CD86. HuABC2, HuABC2-CTLA4 and HuABC2-CTLA4YE bound to CD122 in a dose-dependent manner (FIG. 3). No significant difference was noticed among their binding patterns to CD122. For CD80, HuABC2 showed only the background level of binding while HuABC2-CTLA4 and HuABC2-CTLA4YE bound to CD80 in a dose-dependent manner (FIG. 4). Similarly, for CD86, HuABC2 showed only the background level of binding while HuABC2-CTLA4 and HuABC2-CTLA4YE bound to CD86 in a dose-dependent manner (FIG. 5). Although both HuABC2-CTLA4 and HuABC2-CTLA4YE gained the ability to bind to CD80 and CD86 in addition to the HuABC2-derived ability to bind to CD122, HuABC2-CTLA4YE showed stronger binding to each of CD80 and CD86 than HuABC2-CTLA4 did (FIGS. 4 and 5).

### EXAMPLE 4

### Fusion of CTLA-4 at the C-Terminus of HuABC2 Light Chain

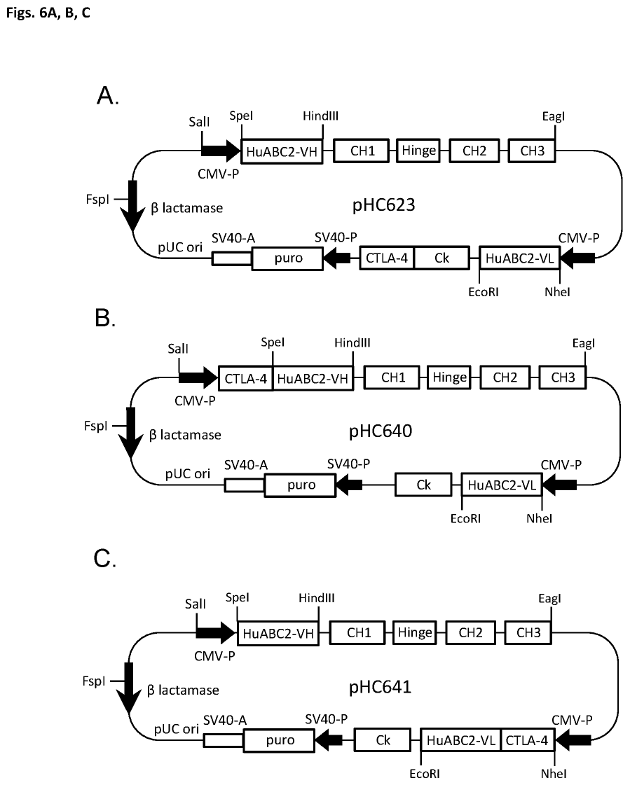

To generate another form of HuABC2 fused to CTLA-4, the Ck exon in pHuABC2p (FIG. 1A) was modified to encode the extracellular domain of the variant form of human CTLA-4 (variant CTLA-4; SEQ ID NO: 14) fused at its N-terminus to the last amino acid residue (Cys) of Ck (Ck-CTLA4YE fusion). A flexible polypeptide linker (SGGGGSG; SEQ ID NO:11) was placed between Ck and CTLA-4 in Ck-CTLA4YE fusion. The resulting expression vector derived from pHuABC2p was termed pHC623. Leucine residues at positions 234 and 235 (EU numbering) in CH2 of pHC623 were both changed to alanine residues to eliminate binding to Fc gamma receptors (Hezareh et al., J. Virol. 75: 12161-12168, 2001). The schematic structure of pHC623 is shown in FIG. 6A.

Amino acid sequence of the Ck-CTLA4YE fusion encoded in pHC623 is

(SEQ ID NO: 32)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGECSGGGGSGMHVAQPAVVLASSRGIASFVCEYASPGKYTEVRVTV
LRQADSQVTEVCAATYMMGNELTFLDDSICTGTSSGNQVNLTIQGLRAMD
TGLYICKVELMYPPPYYEGIGNGTQIYVIDPEPCPDSD.

The linker sequence is underlined.

Amino acid sequence of the mature heavy chain encoded in pHC623 is

(SEQ ID NO: 33)
EVQLVESGGGLVQPGGSLRLSCAASGFTFSDFYMEWVRQAPGKGLEWIAA
SRNKANDYTTEYSASVKGRFIVSRDDSKNSLYLQMNSLKTEDTAVYYCAR
SYYRYDGMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K.

Amino acid sequence of the mature light chain of HuABC2 fused at the C-terminus to the linker (underlined) followed by the extracellular domain of the variant CTLA-4 encoded in pHC623 is

(SEQ ID NO: 34)
EIVLTQSPATLSLSPGERATLSCSAISSVSYMYWYQQKPGQAPRLLIYDT
SNLVSGVPARFSGSGSGTDYTLTISSLEPEDFAVYYCQQWNTYPYTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGECSGGGGSGMHVAQPAVVLASSRGIASFVCEYASPGKYT
EVRVTVLRQADSQVTEVCAATYMMGNELTFLDDSICTGTSSGNQVNLTIQ
GLRAMDTGLYICKVELMYPPPYYEGIGNGTQIYVIDPEPCPDSD.

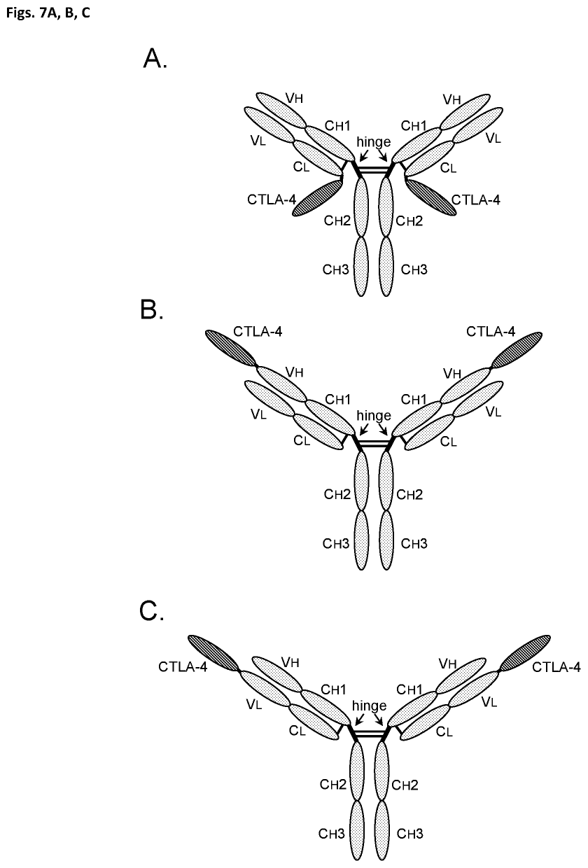

Modified HuABC2 fused to the variant CTLA-4 at the end of the light chain, which is encoded in pHC623, was termed HuABC2-Ck-CTLA4YE. The schematic structure of HuABC2-Ck-CTLA4YE is shown in FIG. 7A.

### EXAMPLE 5

### Fusion of CTLA-4 at the N-Terminus of HuABC2 Heavy Chain

An alternative form of HuABC2 fused to CTLA-4 was constructed by modifying the VH exon encoding HuABC2 VH in pHuABC2p. The variant CTLA-4 (SEQ ID NO: 14) preceded by a synthetic signal peptide (MGWSWIFFFLLSGTASVLS; SEQ ID NO:35) and followed by a flexible polypeptide linker (SGGGGSGGGGS; SEQ ID NO:36) was fused to the N-terminus of mature HuABC2 VH (SEQ ID NO:37) to generate CTLA-4-HuABC2 VH fusion (CTLA4YE-VH fusion). The amino acid sequence of the CTLA4YE-VH fusion is

(SEQ ID NO: 38)
MGWSWIFFFLLSGTASVLSMHVAQPAVVLASSRGIASFVCEYASPGKYTE
VRVTVLRQADSQVTEVCAATYMMGNELTFLDDSICTGTSSGNQVNLTIQG
LRAMDTGLYICKVELMYPPPYYEGIGNGTQIYVIDPEPCPDSDSGGGGSG
GGGSEVQLVESGGGLVQPGGSLRLSCAASGFTFSDFYMEWVRQAPGKGLE
WIAASRNKANDYTTEYSASVKGRFIVSRDDSKNSLYLQMNSLKTEDTAVY
YCARSYYRYDGMDYWGQGTTVTVSS.

The linker sequence is underlined. Mature CTLA4YE-VH fusion starts at position 20 of SEQ ID NO:38.

The resulting expression vector derived from pHuABC2p was termed pHC640. Leucine residues at positions 234 and 235 (EU numbering) in CH2 of pHC640 are both changed to alanine residues to eliminate binding to Fc gamma receptors. The schematic structure of pHC640 is shown in FIG. 6B.

Amino acid sequence of the mature heavy chain of HuABC2 fused at the N-terminus to the extracellular domain of the variant CTLA-4 followed by the linker (underlined) encoded in pHC640 is

(SEQ ID NO: 39)
MHVAQPAVVLASSRGIASFVCEYASPGKYTEVRVTVLRQADSQVTEVCAA
TYMMGNELTFLDDSICTGTSSGNQVNLTIQGLRAMDTGLYICKVELMYPP
PYYEGIGNGTQIYVIDPEPCPDSDSGGGGSGGGGSEVQLVESGGGLVQPG
GSLRLSCAASGFTFSDFYMEWVRQAPGKGLEWIAASRNKANDYTTEYSAS
VKGRFIVSRDDSKNSLYLQMNSLKTEDTAVYYCARSYYRYDGMDYWGQGT
TVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLT
VDKSRWQQGNVFSCSVMHEALHNHYTQKSLSLSPGK.

Amino acid sequence of the mature light chain encoded in pHC640 is same as the mature light chain sequence encoded in pHuABC2p (SEQ ID NO:9).

Modified HuABC2 fused to the variant CTLA-4 at the N-terminus of mature heavy chain, which is encoded in pHC640, was termed HuABC2-CTLA4YE-VH. The schematic structure of HuABC2-CTLA4YE-VH is shown in FIG. 7B.

### EXAMPLE 6

### Fusion of CTLA-4 at the N-Terminus of HuABC2 Light Chain

An additional form of HuABC2 fused to CTLA-4 was constructed by modifying the Vk exon encoding HuABC2 VL in pHuABC2p. The variant CTLA-4 (SEQ ID NO:14) preceded by a synthetic signal peptide (SEQ ID NO:35) and followed by a flexible polypeptide linker (SGGGGSGGGGS; SEQ ID NO:36) was fused to the N-terminus of mature HuABC2 VL (SEQ ID NO:40) to generate CTLA-4-HuABC2 VL fusion (CTLA4YE-Vk fusion). The amino acid sequence of the CTLA4YE-Vk fusion is

(SEQ ID NO: 41)
MGWSWIFFFLLSGTASVLSMHVAQPAVVLASSRGIASFVCEYASPGKYTE
VRVTVLRQADSQVTEVCAATYMMGNELTFLDDSICTGTSSGNQVNLTIQG
LRAMDTGLYICKVELMYPPPYYEGIGNGTQIYVIDPEPCPDSDSGGGGSG
GGGSEIVLTQSPATLSLSPGERATLSCSAISSVSYMYWYQQKPGQAPRLL
IYDTSNLVSGVPARFSGSGSGTDYTLTISSLEPEDFAVYYCQQWNTYPYT
FGGGTKVEIK.

The linker sequence is underlined. Mature CTLA4YE-Vk fusion starts at position 20 of SEQ ID NO:41.

The resulting expression vector derived from pHuABC2p was termed pHC641. Leucine residues at positions 234 and 235 (EU numbering) in CH2 of pHC641 are both changed to alanine residues to eliminate binding to Fc gamma receptors. The schematic structure of pHC641 is shown in

FIG. 6C.

Amino acid sequence of mature heavy chain encoded in pHC641 is same as the mature heavy chain sequence encoded in pHC623 (SEQ ID NO:33)

Amino acid sequence of the mature light chain of HuABC2 fused at the N-terminus to the extracellular domain of the variant CTLA-4 followed by the linker (underlined) encoded in pHC641 is

(SEQ ID NO: 42)
MHVAQPAVVLASSRGIASFVCEYASPGKYTEVRVTVLRQADSQVTEVCAA
TYMMGNELTFLDDSICTGTSSGNQVNLTIQGLRAMDTGLYICKVELMYPP
PYYEGIGNGTQIYVIDPEPCPDSDSGGGGSGGGGSEIVLTQSPATLSLSP
GERATLSCSAISSVSYMYWYQQKPGQAPRLLIYDTSNLVSGVPARFSGSG
SGTDYTLTISSLEPEDFAVYYCQQWNTYPYTFGGGTKVEIKRTVAAPSVF
IFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC.

Modified HuABC2 fused to the variant CTLA-4 at the N-terminus of mature light chain, which is encoded in pHC641, was termed HuABC2-CTLA4YE-Vk. The schematic structure of HuABC2-CTLA4YE-Vk is shown in FIG. 7C.

### EXAMPLE 7

### Expression and Purification of HuABC2 Fused to CTLA-4

CHO-K1 stable transfectants with each of the five expression vectors (pHuABC2p, pHC622, pHC623, pHC640 and pHC641) were generated as described above. Antibody expression in culture supernatants was analyzed by sandwich ELISA as described above. CHO-K1 stable transfectants producing high level of each of HuABC2, HuABC2-CTLA4YE (also called HuABC2-CH3-CTLA4YE), HuABC2-Ck-CTLA4YE, HuABC2-CTLA4YE-VH and HuABC2-CTLA4YE-Vk antibodies were expanded in SFM4CHO. HuABC2 and its CTLA-4 fusions were purified from culture supernatants by protein A column chromatography as described above.

Purified HuABC2, HuABC2-CH3-CTLA4YE, HuABC2-Ck-CTLA4YE, HuABC2-CTLA4YE-VH and HuABC2-CTLA4YE-Vk were characterized by SDS-PAGE under reducing conditions according to standard procedures. Each of these antibodies was found to be composed of two major polypeptides. Their approximate sizes were 50 kD and 25 kD for HuABC2, 65 kD and 25 kD for HuABC2-CH3-CTLA4YE, 50 kD and 40 kD for HuABC2-Ck-CTLA4YE, 65 kD and 25 kD for HuABC2-CTLA4YE-VH, and 50 kD and 40 kD for HuABC2-CTLA4YE-Vk.

### EXAMPLE 8

### Binding of HuABC2 Fused to CTLA-4 to CD80, CD86 and CD122

To analyze the property of HuABC2-CH3-CTLA4YE, HuABC2-Ck-CTLA4YE, HuABC2-CTLA4YE-VH and HuABC2-CTLA4YE-Vk, two expression vectors (pFCm553 and pFCm554) were constructed for expression of recombinant human CD80 and CD86, respectively, on the cell surface.

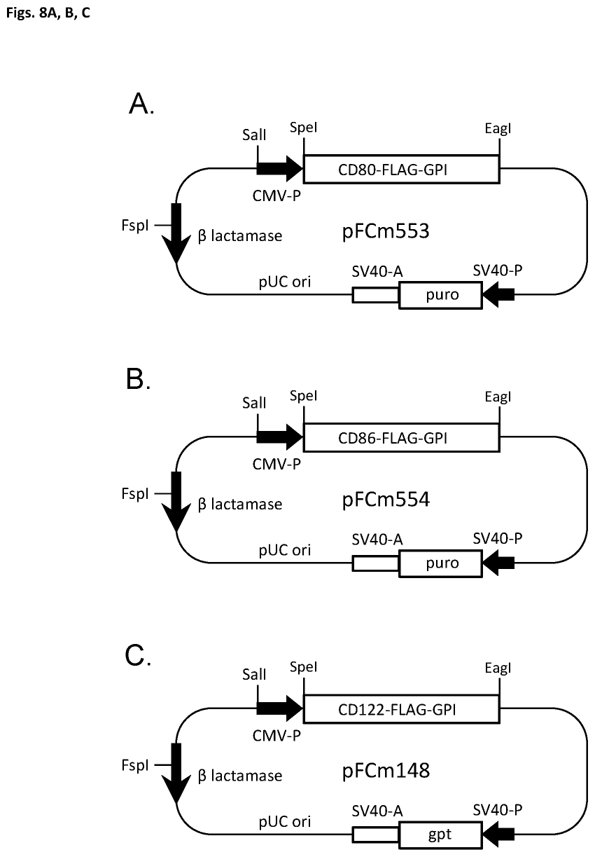

The expression vector pFCm553 (FIG. 8A) has the same structure as pHuABC2p except that (a) the light chain transcription unit was removed and (b) the Spel-Eagl fragment encoding the heavy chain of HuABC2 was substituted with a DNA fragment encoding a synthetic signal peptide (SEQ ID NO:35) followed by the extra cellular region of human CD80 (SEQ ID NO:43), the FLAG polypeptide (DYKDDDDK; SEQ ID NO:44) and the GPI anchorage signal derived from human CD55 (PNKGSGTTSGTTRLLSGHTCFTLTGLLGTLVTMGLLT; SEQ ID NO:45) (CD80-FLAG-GPI). The amino acid sequence of mature CD80-FLAG-GPI is

(SEQ ID NO: 46)
VIHVTKEVKEVATLSCGHNVSVEELAQTRIYWQKEKKMVLTMMSGDMNIW
PEYKNRTIFDITNNLSIVILALRPSDEGTYECVVLKYEKDAFKREHLAEV
TLSVKADFPTPSISDFEIPTSNIRRIICSTSGGFPEPHLSWLENGEELNA
INTTVSQDPETELYAVSSKLDFNMTTNHSFMCLIKYGHLRVNQTFNWNTT
KQEHFPDNTGGGDYKDDDDKGGGPNKGSGTTSGTTRLLSGHTCFTLTGLL
GTLVTMGLLT.

In another expression vector pFCm554 (FIG. 8B), the CD80-coding region in pFCm553 is replaced by the coding region of the extracellular domain of human CD86 (SEQ ID NO:47) to express human CD86 fused to the FLAG peptide and GPI anchorage signal (CD86-FLAG-GPI). The amino acid sequence of mature CD86-FLAG-GPI is

(SEQ ID NO: 48)
LKIQAYFNETADLPCQFANSQNQSLSELVVFWQDQENLVLNEVYLGKEKF
DSVHSKYMGRTSFDSDSVVTLRLHNLQIKDKGLYQCIIHHKKPTGMIRIH
QMNSELSVLANFSQPEIVPISNITENVYINLTCSSIHGYPEPKKMSVLLR
TKNSTIEYDGIMQKSQDNVTELYDVSISLSVSFPDVTSNMTIFCILETDK
TRLLSSPFSIELEDPQPPPDHTGGGDYKDDDDKGGGPNKGSGTTSGTTRL
LSGHTCFTLTGLLGTLVTMGLLT.

CHO-K1 stable transfectants expressing CD80-FLAG-GPI (CHO-K1/CD80) and CD86-FLAG-GPI (CHO-K1/CD86) were generated by electroporation with pFCm553 and pFCm554, respectively, as described above. Binding of HuABC2-CH3-CTLA4YE, HuABC2-Ck-CTLA4YE, HuABC2-CTLA4YE-VH and HuABC2-CTLA4YE-Vk to CHO-K1/CD80 and CHO-K1/CD86 cells was analyzed by flow cytometry. Belatacept (also known as LEA29Y and Nulojix; SEQ ID NO:49), which is a CTLA4-Ig fusion protein carrying a substitution of an alanine residue to a tyrosine residue at position 29 (A29Y) and another substitution of a leucine residue to a glutamate residue at position 104 (L104E) of SEQ ID NO:10 (Larsen et al., Am. J. Transplant. 5: 443-453, 2005; Oshima et al., Protein Eng. Des. Sel. 29: 159-167, 2016), was purchased from Creative Biomart (Cat. No. THP-0109, Shirley, NY) and used as a reference in the experiments.

Purified test samples (HuABC2-CH3-CTLA4YE, HuABC2-Ck-CTLA4YE, HuABC2-CTLA4YE-VH, and HuABC2-CTLA4YE-Vk) and belatacept, starting at 10 μg/ml and five steps of 4-fold serial dilutions, were incubated with CHO-K1/CD80 or CHO-K1/CD86 cells in FACS Buffer (PBS containing 0.5% BSA and 0.05% sodium azide) for 30 min at 4° C. After washing with FACS Buffer, cells were incubated with PE-labeled goat anti-human IgG antibody (SouthernBiotech, Birmingham, AL) in FACS Buffer for 20 min at 4° C. After washing and suspension in FACS Buffer, stained cells were subjected to flow cytometry.

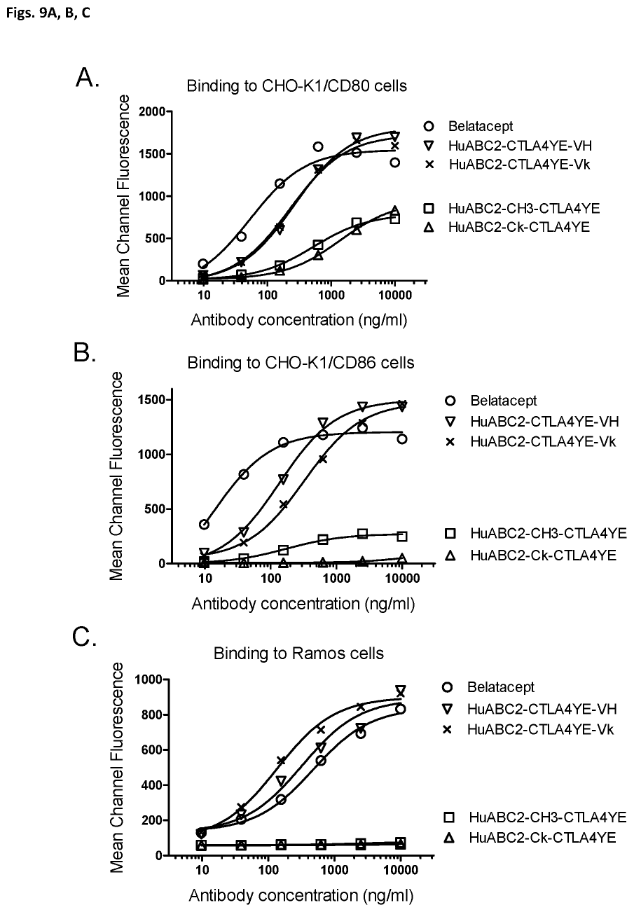

The binding patterns of HuABC2-CH3-CTLA4YE, HuABC2-Ck-CTLA4YE, HuABC2-CTLA4YE-VH, HuABC2-CTLA4YE-Vk and belatacept to CHO-K1/CD80 cells are shown in FIG. 9A. While the binding of each of HuABC2-CTLA4YE-VH and HuABC2-CTLA4YE-Vk to CD80 was only slightly weaker than the binding of belatacept, the binding of HuABC2-CH3-CTLA4YE and HuABC2-Ck-CTLA4YE was a lot weaker than that of belatacept. For example, the mean channel fluorescence (MCF) values for binding to CHO-K1/CD80 cells at 625 ng/ml were 1,583 for belatacept, 425 for HuABC2-CH3-CTLA4YE, 307 for HuABC2-Ck-CTLA4YE, 1,313 for HuABC2-CTLA4YE-VH and 1,310 for HuABC2-CTLA4YE-Vk. Provided that the molecular weight of belatacept is approximately 90 kilodaltons (kD) according to the package insert of Nulojix (belatacept; Bristol Myers Squibb), which is roughly half of the molecular weight of HuABC2-CTLA4YE-VH and HuABC2-CTLA4YE-Vk (both approximately 180 kD), the binding of HuABC2-CTLA4YE-VH and HuABC2-CTLA4YE-Vk, in which CTLA-4 is fused to the N-terminus of heavy and light chains, respectively, to CD80 was comparable to that of belatacept on a molar basis. In contrast, the binding of HuABC2-CH3-CTLA4YE and HuABC2-Ck-CTLA4YE, in which CTLA-4 was fused to the C-terminus of heavy and light chains, respectively, was unexpectedly much weaker than that of belatacept.

The binding patterns of HuABC2-CH3-CTLA4YE, HuABC2-Ck-CTLA4YE, HuABC2-CTLA4YE-VH, HuABC2-CTLA4YE-Vk and belatacept to CHO-K1/CD86 cells are shown in FIG. 9B. Like the case with CHO-K1/CD80 cells, while the binding of each of HuABC2-CTLA4YE-VH and HuABC2-CTLA4YE-Vk to CD86 was only slightly weaker than the binding of belatacept, the binding of HuABC2-CH3-CTLA4YE and HuABC2-Ck-CTLA4YE was a lot weaker than the binding of belatacept. For example, the MCF values for binding to CHO-K1/CD86 cells at 625 ng/ml were 1,179 for belatacept, 221 for HuABC2-CH3-CTLA4YE, 13 for HuABC2-Ck-CTLA4YE, 1,288 for HuABC2-CTLA4YE-VH and 956 for HuABC2-CTLA4YE-Vk. The binding of HuABC2-CTLA4YE-VH and HuABC2-CTLA4YE-Vk to CD86 was comparable to the binding of belatacept on a molar basis, whereas the binding of HuABC2-CH3-CTLA4YE and HuABC2-Ck-CTLA4YE was again unexpectedly much weaker than that of belatacept.

Binding property of HuABC2-CH3-CTLA4YE, HuABC2-Ck-CTLA4YE, HuABC2-CTLA4YE-VH, HuABC2-CTLA4YE-Vk and belatacept was further examined with human Burkitt's lymphoma cell line Ramos by flow cytometry as described above. The result is shown in FIG. 9C. The MCF values for binding to Ramos cells at 625 ng/ml were 539 for belatacept, 63 for HuABC2-CH3-CTLA4YE, 60 for HuABC2-Ck-CTLA4YE, 611 for HuABC2-CTLA4YE-VH and 715 for HuABC2-CTLA4YE-Vk. As observed with CHO-K1/CD80 and CHO-K1/CD86 cells, the binding of HuABC2-CTLA4YE-VH and HuABC2-CTLA4YE-Vk to Ramos cells was comparable to the binding of belatacept, while the binding of HuABC2-CH3-CTLA4YE and HuABC2-Ck-CTLA4YE was much weaker than that of belatacept.

### EXAMPLE 9

### Modification of the Linker Between CH3 and CTLA-4

HuABC2-CH3-CTLA4YE showed weak binding to CD80 and CD86 when compared to belatacept and HuABC2-CTLA4YE-VH (FIGS. 9A and B). To assess the influence of a flexible polypeptide linker between CH3 and CTLA-4 on binding to CD80 and CD86, two new expression vectors were generated by modifying the sequence of the polypeptide linker (SGGGGSG; SEQ ID NO:11) connecting CH3 and CTLA-4 in pHC625 encoding HuABC2-CH3-CTLA4YE. The sequence of the polypeptide linker between CH3 and CTLA-4 in one of the resulting vectors, pHC634, is SGGGGSGGGGS (SEQ ID NO:36). The modified HuABC2-CH3-CTLA4YE expressed from pHC634 was termed HuABC2-CH3-CTLA4YE-X. The sequence of the polypeptide linker between CH3 and CTLA-4 in the other resulting vector, pHC635, is SGGGGSGGGGSGGGGS (SEQ ID NO:50). The modified HuABC2-CH3-CTLA4YE expressed from pHC635 was termed HuABC2-CH3-CTLA4YE-Y.

Each of the three expression vectors (pHC625, pHC634 and pHC635) was transfected into HEK293 cells as described above. The expression level of HuABC2-CH3-CTLA4YE, HuABC2-CH3-CTLA4YE-X and HuABC2-CH3-CTLA4YE-Y in culture supernatants was measured by sandwich ELISA as described above. Binding of HuABC2-CH3-CTLA4YE, HuABC2-CH3-CTLA4YE-X and HuABC2-CH3-CTLA4YE-Y to each of CHO-K1/CD80 and CHO-K1/CD86 cells was analyzed by flow cytometry as described above. HuABC2-CH3-CTLA4YE, HuABC2-CH3-CTLA4YE-X and HuABC2-CH3-CTLA4YE-Y showed no clear difference in binding to CD80 and CD86.

### EXAMPLE 10

### Binding to CD122

To assess the possibility that the attachment of CTLA-4 at the N-terminus of heavy or light chain of HuABC2 interferes with its antigen binding, the ability of HuABC2-CTLA4YE-VH and HuABC2-CTLA4YE-Vk to bind to CD122 was compared to that of HuABC2. An expression vector pFCm148 (FIG. 8C) has the same structure as pFCm553 (FIG. 8A) except that the coding regions of CD80 and puromycin N-acetyl-transferase are substituted by the extracellular domain of human CD122 (SEQ ID NO:51) and xanthine-guanine phosphoribosyltransferase (shown as gpt in FIG. 8C), respectively. The amino acid sequence of mature human CD122 fused to the FLAG peptide and GPI anchorage signal (CD122-FLAG-GPI) encoded in pFCm148 is

(SEQ ID NO: 52)
AVNGTSQFTCFYNSRANISCVWSQDGALQDTSCQVHAWPDRRRWNQTCEL
LPVSQASWACNLILGAPDSQKLTTVDIVTLRVLCREGVRWRVMAIQDFKP
FENLRLMAPISLQVVHVETHRCNISWEISQASHYFERHLEFEARTLSPGH
TWEEAPLLTLKQKQEWICLETLTPDTQYEFQVRVKPLQGEFTTWSPWSQP
LAFRTKPAALGKDTTGGGDYKDDDDKGGGPNKGSGTTSGTTRLLSGHTCF
TLTGLLGTLVTMGLLT.

A mouse myeloma cell line NSO was stably transfected by electroporation with pFCm148 and selected for expression of xanthine-guanine phosphoribosyltransferase according to standard procedures. An NSO stable transfectant expressing CD122-FLAG-GPI on the surface (NS0/CD122) was used for the analysis of binding of HuABC2, HuABC2-CTLA4YE-VH and HuABC2-CTLA4YE-Vk to CD122.

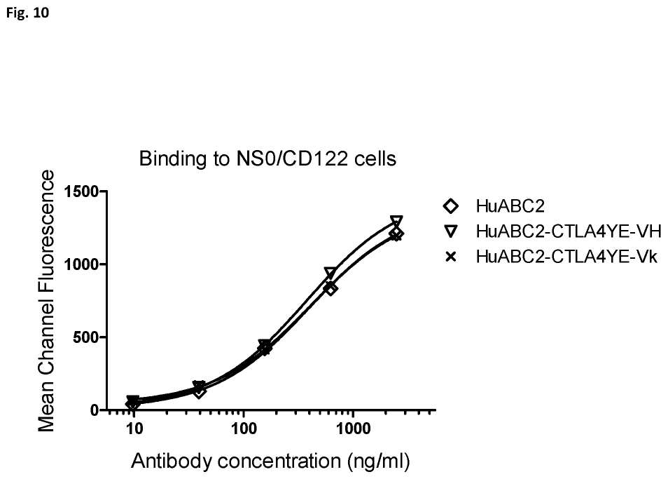

Each of HuABC2, HuABC2-CTLA4YE-VH and HuABC2-CTLA4YE-Vk, starting at 2.5 μg/ml and four steps of 4-fold serial dilutions, was incubated with NSO/CD122 cells in FACS Buffer for 30 min at 4° C. After washing with FACS Buffer, cells were incubated with PE-labeled goat anti-human IgG antibody for 20 min at 4° C. After washing and suspension in FACS Buffer, cells were subjected to flow cytometry. As shown in FIG. 10, HuABC2, HuABC2-CTLA4YE-VH and HuABC2-CTLA4YE-Vk showed no clear difference for binding to CD122. Thus, antigen binding of HuABC2 was not negatively affected by fusion of CTLA-4 to the N-terminus of its heavy or light chain.

### EXAMPLE 11

### Immunosuppressive Activity of HuABC2 Fused to CTLA-4

The activity of HuABC2 fused to CTLA-4 to suppress immune responses was examined by a mixed lymphocyte reaction (MLR) using CD3+ T cells and allogenic monocyte-derived dendritic cells (Mo-DCs) (Latek et al., Transplantation, 87: 926-933, 2009; Goenka et al., J. Immunol, 206: 1102-1113, 2021). CD3+ T cells were purified from human peripheral blood mononuclear cells (PBMC) using the MojoSort Human CD3 T Cell Isolation Kit (BioLegend, San Diego, CA) and labeled with 2.5 μM CellTrace Far Red (Thermo Fisher Scientific). To generate mature Mo-DCS, CD14+ monocytes were isolated from human PBMC of a different donor using CD14 MicroBeads, human (Miltenyi Biotec, Auburn, CA), cultured in RPMI 1640 medium supplemented with 10% FBS (Life Technologies, Grand Islands, NY), 10 mM HEPES, 1 mM sodium pyruvate, 250 IU/ml human IL-4 (BioLegend) and 800 IU/ml human GM-CSF (BioLegend) for 6 days at 37° C. in a 7.5% CO₂ incubator, and further grown for 1 day in RPMI 1640 medium supplemented with 10% FBS, 10 mM HEPES, 1 mM sodium pyruvate, 1,000 IU/ml human IL-6 (BioLegend), 200 IU/ml human IL-1β (BioLegend) and 1,000 IU/ml human TNF-α (BioLegend).

MLR was conducted by mixing CD3+ T cells labeled with CellTrace Far Red and allogeneic mature Mo-DCs at a 2:1 ratio in RPMI 1640 medium supplemented with 10% FBS, 10 mM HEPES, 1 mM sodium pyruvate in the presence or absence of 3 μg/ml of each test sample or their combination. After 6-day incubation at 37° C. in a 7.5% CO₂ incubator, cells were stained with Helix NP Green (BioLegend) and subjected to flow cytometry. Fluorescence levels of Helix NP Green and CellTrace Far Red were used to monitor viability and proliferation of cells, respectively. Mo-DCS, which were not stained by CellTrace Far Red and thus negative for its fluorescence, were excluded from the analysis. T cell proliferation was monitored by the level of fluorescence of CellTrace Far Red according to the User Guide of the CellTrace Far Red Cell Proliferation Kit (Thermo Fisher Scientific) and calculated by dividing the number of proliferating T cells by the number of total live T cells.

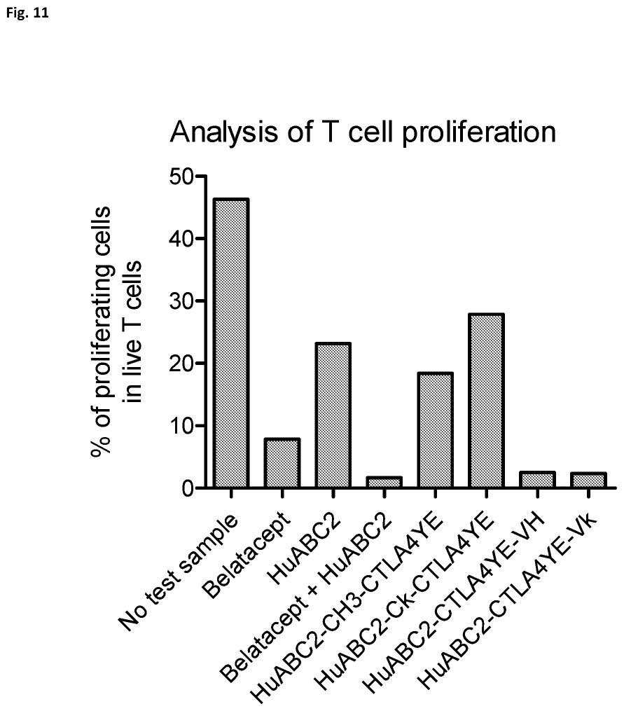

The result of the analysis of T cell proliferation in MLR is shown in FIG. 11. The ratio of proliferating T cells in the live T cell population was 46.3% with no test sample, 7.9% with belatacept, 23.2% with HuABC2, 1.7% with a combination of belatacept and HuABC2, 18.4% with HuABC2-CH3-CTLA4YE, 27.9% with HuABC2-Ck-CTLA4YE, 2.5% with HuABC2-CTLA4YE-VH, and 2.4% with HuABC2-CTLA4-Vk. Both HuABC2-CTLA4YE-VH and HuABC2-CTLA4-Vk suppressed T cell proliferation more potently than each of belatacept and HuABC2, and nearly as efficiently as the combination of HuABC2 and belatacept. The activity of each of HuABC2-CH3-CTLA4YE and HuABC2-Ck-CTLA4YE to suppress T cell proliferation was much weaker than the activity of HuABC2-CTLA4YE-VH and HuABC2-CTLA4-Vk.

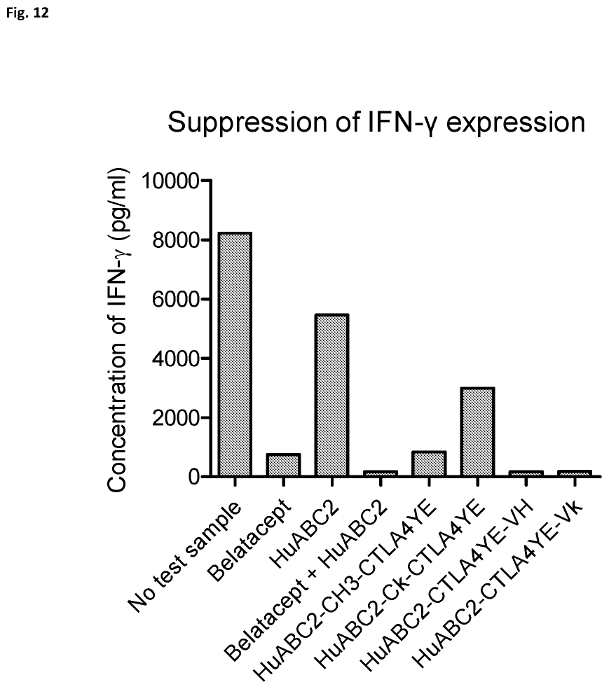

The level of proinflammatory cytokine IFN-γ in MLR culture supernatants measured using the ELISA MAX Deluxe Set Human IFN-γ kit (BioLegend) was 8,217 pg/ml with no test sample, 744 pg/ml with belatacept, 5,459 pg/ml with HuABC2, 170 pg/ml with a combination of belatacept and HuABC2, 837 pg/ml with HuABC2-CH3-CTLA4YE, 2,984 pg/ml with HuABC2-Ck-CTLA4YE, 167 pg/ml with HuABC2-CTLA4YE-VH, and 182 pg/ml with HuABC2-CTLA4YE-Vk (FIG. 12). Both HuABC2-CTLA4YE-VH and HuABC2-CTLA4YE-Vk suppressed the production of IFN-γ as potently as the combination of HuABC2 and belatacept.

### EXAMPLE 12

### HuABC101 Fused to CTLA-4

HuABC101, which is expressed from the vector pHuABC101 in mammalian cells, is a humanized anti-CD122 IgG1/kappa antibody comprising HuABC101 VH (SEQ ID NO:29) and HuABC101 VL (SEQ ID NO:30). The structure of pHuABC101 is same as pHuABC2p (FIG. 1A) except that the coding regions of HuABC2 VH, HuABC2 VL, and puromycin N-acetyl-transferase are replaced by HuABC101 VH, HuABC101 VL, and xanthine-guanine phosphoribosyltransferase, respectively.

Amino acid sequence of the mature heavy chain encoded in pHuABC101 is

(SEQ ID NO: 53)
QVQLQESGPGLVKPSQTLSLTCTVSGYSITNDNHWWNWIRQHPGKGLEWM
GYIDSSGSSDNNPSLKSQITISRDTSKNQLSLKLSSVTAADTAVYYCARG
GGRDYYGMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K.

Amino acid sequence of the mature light chain encoded in HuABC101 is DIVMTQSPDSLGVSLGERATINCRASQSVSTSSYSYVHWYQQKPGQPPKLLIKYASNLESGVPDRFSGSGSGTDFTLTISS LQAEDVAVYYCQHSWDIPFTFGQGTKLEIKRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGEC (SEQ ID NO:54).

The variant CTLA-4 (SEQ ID NO: 14) was fused to the N-terminus of mature HuABC101 VH in pHuABC101 to generate a new expression vector pHC642. The coding region of CTLA-4 fused to HuABC101 VH in pHC642 comprises, from the N-to C-terminus, a synthetic signal peptide (SEQ ID NO:35), variant CTLA-4 (SEQ ID NO:14), a flexible polypeptide linker (SGGGGSGGGGS; SEQ ID NO:36), and mature HuABC101 VH (SEQ ID NO:29) (CTLA4YE-HuABC101 VH fusion). The amino acid sequence of the CTLA4YE-HuABC101 VH fusion is

(SEQ ID NO: 55)
MGWSWIFFFLLSGTASVLSMHVAQPAVVLASSRGIASFVCEYASPGKYTE
VRVTVLRQADSQVTEVCAATYMMGNELTFLDDSICTGTSSGNQVNLTIQG
LRAMDTGLYICKVELMYPPPYYEGIGNGTQIYVIDPEPCPDSDSGGGGSG
GGGSQVQLQESGPGLVKPSQTLSLTCTVSGYSITNDNHWWNWIRQHPGKG
LEWMGYIDSSGSSDNNPSLKSQITISRDTSKNQLSLKLSSVTAADTAVYY
CARGGGRDYYGMDYWGQGTTVTVSS.

The linker sequence is underlined. Mature CTLA4YE-HuABC101 VH fusion starts at position 20 of SEQ ID NO:55.

Amino acid sequence of the mature light chain encoded in pHC642 is same as the mature light chain sequence encoded in pHuABC101 (SEQ ID NO:54). Amino acid sequence of the mature heavy chain of HuABC101 fused at the N-terminus to the extracellular domain of the variant CTLA-4 followed by the linker (underlined) encoded in pHC642 is

(SEQ ID NO: 56)
MHVAQPAVVLASSRGIASFVCEYASPGKYTEVRVTVLRQADSQVTEVCAA
TYMMGNELTFLDDSICTGTSSGNQVNLTIQGLRAMDTGLYICKVELMYPP
PYYEGIGNGTQIYVIDPEPCPDSDSGGGGSGGGGSQVQLQESGPGLVKPS
QTLSLTCTVSGYSITNDNHWWNWIRQHPGKGLEWMGYIDSSGSSDNNPSL
KSQITISRDTSKNQLSLKLSSVTAADTAVYYCARGGGRDYYGMDYWGQGT
TVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLT
VDKSRWQQGNVFSCSVMHEALHNHYTQKSLSLSPGK.

Modified HuABC101 with fusion to CTLA-4 at the N-terminus of heavy chain, which was termed HuABC101-CTLA4YE-VH, is expressed from pHC642.

Another expression vector was constructed by fusing a flexible polypeptide linker (SEQ ID NO:11) followed by the variant CTLA-4 (SEQ ID NO: 14) to the penultimate glycine residue of CH3 in pHuABC101. Amino acid sequence of the mature light chain encoded in the resulting vector pHC630 is same as the mature light chain sequence encoded in pHuABC101 (SEQ ID NO:54). Amino acid sequence of the mature heavy chain of HuABC101 fused at the C-terminus to the linker (underlined) followed by the extracellular domain of the variant CTLA-4 encoded in pHC630 is

(SEQ ID NO: 57)
QVQLQESGPGLVKPSQTLSLTCTVSGYSITNDNHWWNWIRQHPGKGLEWM
GYIDSSGSSDNNPSLKSQITISRDTSKNQLSLKLSSVTAADTAVYYCARG
GGRDYYGMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
SGGGGSGMHVAQPAVVLASSRGIASFVCEYASPGKYTEVRVTVLRQADSQ
VTEVCAATYMMGNELTFLDDSICTGTSSGNQVNLTIQGLRAMDTGLYICK
VELMYPPPYYEGIGNGTQIYVIDPEPCPDSD.

Modified HuABC101 with fusion to CTLA-4 at the C-terminus of heavy chain, which was termed HuABC101-CH3-CTLA4YE, is expressed from pHC630.

The two expression vectors (pHC642 and pHC630) were individually transfected into HEK293 cells as described above. Their expression level in culture supernatants was measured by sandwich ELISA as described above. Binding of transiently expressed HuABC101-CTLA4YE-VH and HuABC101-CH3-CTLA4YE to CD80 and CD86 was analyzed by flow cytometry as described above. Belatacept was used as a reference for binding to CD80 and CD86.

The MCF values for binding of HuABC101-CTLA4YE-VH, HuABC101-CH3-CTLA4YE and belatacept, each at 200 ng/ml, to CHO-K1/CD80 cells were 1163, 291 and 1876, respectively. The MCF values for binding of HuABC101-CTLA4YE-VH, HuABC101-CH3-CTLA4YE and belatacept, each at 200 ng/ml, to CHO-K1/CD86 cells were 745, 108 and 917, respectively. Binding of HuABC101-CH3-CTLA4YE to each of CD80 and CD86 was much weaker than the binding of HuABC101-CTLA4YE-VH. As observed with HuABC2 fused to CTLA-4, fusion of CTLA-4 to the C-terminus of heavy chain of HuABC101 unexpectedly reduced the binding to CD80 and CD86.

### EXAMPLE 13

### HuABC77D Fused to CTLA-4

Mouse hybridoma producing an IgG/kappa monoclonal antibody ABC77 that binds to and blocks the function of human and cynomolgus CD122 was isolated at JN Biosciences (Mountain View, CA) following standard cell fusion techniques using GenomONE CF EX Cell Fusion Reagent (Cosmo Bio, Carlsbad, CA) as described in U.S. Pat. No. 9,028,830. Sequencing and humanization VH and VL of ABC77 were performed as described in Tsurushita et al. (Methods 36: 69-83, 2005).

Amino acid sequence of humanized ABC77 VH (HuABC77D VH) is

(SEQ ID NO: 58)
MDFGLIFFIVALLKGVQCEVQLVESGGGLVQPGGSLRLSCAASGFDFSRY
WMSWVRQAPGKGLEWIGEINPDSSTINYAPSLKDRFTISRDNAKNSLYLQ
MNSLRAEDTAVYYCARHDYDEGIYFDYWGQGTTVTVSS.

Amino acid sequences of CDR1, 2 and 3 of HuABC77D VH based on the definition of Kabat et al. (supra) is RYWMS (SEQ ID NO:59), EINPDSSTINYAPSLKD (SEQ ID NO:60) and HDYDEGIYFDY (SEQ ID NO:61), respectively. Mature HuABC77D VH starts at position 19 of SEQ ID NO:58.

Amino acid sequence of humanized ABC77 VL (HuABC77D VL) is

(SEQ ID NO: 62)
MDFQVQIFSFLLISASVIISRGDIQMTQSPSSLSASVGDRVTITCSASSR
VIYMHWYQQKPGKAPKRWIYDTSKLASGVPSRFSGSGSGTEFTLTISSLQ
PEDFATYYCQQWSSNPPTFGGGTKVEIK.

Amino acid sequences of CDR1, 2 and 3 of HuABC77D VL based on the definition of Kabat et al. (supra) is SASSRVIYMH (SEQ ID NO: 63), DTSKLAS (SEQ ID NO: 64) and QQWSSNPPT (SEQ ID NO:65), respectively. Mature HuABC77D VL starts at position 23 of SEQ ID NO:62.

The expression vector pHuABC77D is a derivative of pHuABC2p, in which the coding regions of HuABC2 VH and VL are replaced by those of HuABC77D VH and VL, respectively. Leucine residues at positions 234 and 235 (EU numbering) in CH2 of pHuABC77D were both changed to alanine residues to eliminate binding to Fc gamma receptors (Hezareh et al., J. Virol. 75: 12161-12168, 2001).

Amino acid sequence of the mature heavy chain encoded in pHuABC77D is

(SEQ ID NO: 66)
EVQLVESGGGLVQPGGSLRLSCAASGFDFSRYWMSWVRQAPGKGLEWIGE
INPDSSTINYAPSLKDRFTISRDNAKNSLYLQMNSLRAEDTAVYYCARHD
YDEGIYFDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

Amino acid sequence of the mature light chain encoded in pHuABC77D is

(SEQ ID NO: 67)
DIQMTQSPSSLSASVGDRVTITCSASSRVIYMHWYQQKPGKAPKRWIYDT
SKLASGVPSRFSGSGSGTEFTLTISSLQPEDFATYYCQQWSSNPPTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

CHO-K1 cells stably transfected with pHuABC77D were generated and expanded in SFM4CHO media as described above. Humanized ABC77 IgG1/kappa antibody (HuABC77D) was purified from culture supernatants as described above. HuABC77D showed (i) specific binding to human and cynomolgus CD122 and (ii) blocking of the interaction of human CD122 with human IL-2 and IL-15.

The variant CTLA-4 (SEQ ID NO: 14) was fused to the N-terminus of mature HuABC77D VH in pHuABC77D to generate a new expression vector pHC644. The coding region of CTLA-4 fused to HuABC77D VH in pHC644 comprises, from the N-to C-terminus, a synthetic signal peptide (SEQ ID NO:35), variant CTLA-4 (SEQ ID NO:14), a flexible polypeptide linker (SGGGGSGGGGS; SEQ ID NO:36), and mature HuABC77D VH (SEQ ID NO:68) (CTLA4YE-HuABC77D VH fusion). The amino acid sequence of mature CTLA4YE-HuABC77D VH fusion is

(SEQ ID NO: 69)
MHVAQPAVVLASSRGIASFVCEYASPGKYTEVRVTVLRQADSQVTEVCAA
TYMMGNELTFLDDSICTGTSSGNQVNLTIQGLRAMDTGLYICKVELMYPP
PYYEGIGNGTQIYVIDPEPCPDSDSGGGGSGGGGSEVQLVESGGGLVQPG
GSLRLSCAASGFDFSRYWMSWVRQAPGKGLEWIGEINPDSSTINYAPSLK
DRFTISRDNAKNSLYLQMNSLRAEDTAVYYCARHDYDEGIYFDYWGQGTT
VTVSS.

The linker sequence is underlined.

Amino acid sequence of the mature light chain encoded in pHC644 is same as the mature light chain sequence encoded in pHuABC77D (SEQ ID NO:67). Amino acid sequence of the mature heavy chain of HuABC77D fused at the N-terminus to the extracellular domain of the variant CTLA-4 followed bv the linker (underlined) encoded in DHC644 is

(SEQ ID NO: 70)
MHVAQPAVVLASSRGIASFVCEYASPGKYTEVRVTVLRQADSQVTEVCAA
TYMMGNELTFLDDSICTGTSSGNQVNLTIQGLRAMDTGLYICKVELMYPP
PYYEGIGNGTQIYVIDPEPCPDSDSGGGGSGGGGSEVQLVESGGGLVQPG
GSLRLSCAASGFDFSRYWMSWVRQAPGKGLEWIGEINPDSSTINYAPSLK
DRFTISRDNAKNSLYLQMNSLRAEDTAVYYCARHDYDEGIYFDYWGQGTT
VTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVD
KKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPGK.

Modified HuABC77D with fusion to CTLA-4 at the N-terminus of heavy chain, which was termed HuABC77D-CTLA4YE-VH, is expressed from pHC644.

Another expression vector was constructed by fusing the variant CTLA-4 (SEQ ID NO:14) to the penultimate glycine residue of CH3 in pHuABC77D. Amino acid sequence of the mature light chain encoded in the resulting vector pHC645 is same as the mature light chain sequence encoded in pHuABC77D (SEQ ID NO:67). Amino acid sequence of the mature heavy chain of HuABC77D fused at the C-terminus to the linker (underlined) followed by the extracellular domain of the variant CTLA-4 encoded in pHC645 is

(SEQ ID NO: 71)
EVQLVESGGGLVQPGGSLRLSCAASGFDFSRYWMSWVRQAPGKGLEWIGE
INPDSSTINYAPSLKDRFTISRDNAKNSLYLQMNSLRAEDTAVYYCARHD
YDEGIYFDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGS
GGGGSGMHVAQPAVVLASSRGIASFVCEYASPGKYTEVRVTVLRQADSQV
TEVCAATYMMGNELTFLDDSICTGTSSGNQVNLTIQGLRAMDTGLYICKV
ELMYPPPYYEGIGNGTQIYVIDPEPCPDSD.

Modified HuABC77D with fusion to CTLA-4 at the C-terminus of heavy chain, which was termed HuABC77D-CH3-CTLA4YE, is expressed from pHC645.

The two expression vectors (pHC644 and pHC645) were individually transfected into HEK293 cells as described above. Their expression level in culture supernatants was measured by sandwich ELISA as described above. Binding of HuABC77D-CH3-CTLA4YE and HuABC77D-CTLA4YE-VH to CHO-K1/CD80 and CHO-K1/CD86 cells was analyzed by flow cytometry as described above. Belatacept was used as a reference for binding to CD80 and CD86. The test samples were used at 500 ng/ml and four steps of 3-fold serial dilutions.

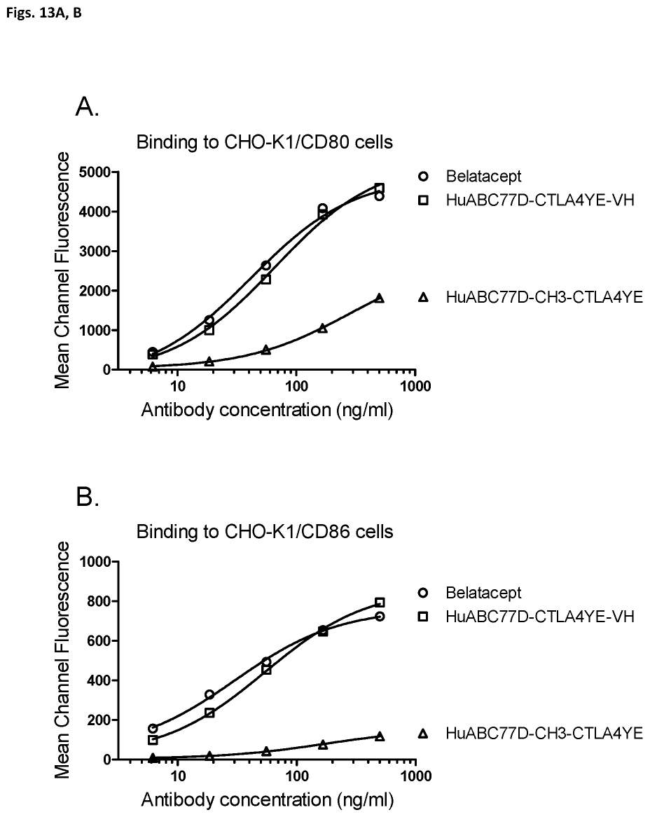

The results of binding to CHO-K1/CD80 and CHO-K1/CD86 cells are shown in FIGS. 13A and B, respectively. Belatacept and HuABC77D-CTLA4YE-VH showed the same level of binding to each of CD80 and CD86, whereas the binding of HuABC77D-CH3-CTLA4YE was much weaker when compared to the binding of belatacept and HuABC77D-CTLA4YE-VH. The MCF values for binding of belatacept, HuABC77D-CTLA4YE-VH and HuABC77D-CH3-CTLA4YE, each at 500 ng/ml, to CHO-K1/CD80 cells were 4390, 4594 and 1809, respectively. The MCF values for binding of belatacept, HuABC77D-CTLA4YE-VH and HuABC77D-CH3-CTLA4YE, each at 500 ng/ml, to CHO-K1/CD86 cells were 723, 794 and 118, respectively. As observed with HuABC2 and HuABC101, fusion of CTLA-4 to the C-terminus of heavy chain unexpectedly reduced the binding of HuABC77D to CD80 and CD86.

Binding of HuABC77D, HuABC77D-CTLA4YE-VH and HuABC77D-CH3-CTLA4YE to CD122 was analyzed by flow cytometry using NSO/CD122 cells as described above. No major difference was observed with the CD122 binding pattern among HuABC77D, HuABC77D-CTLA4YE-VH and HuABC77D-CH3-CTLA4YE. This result indicates that fusion of CTLA-4 at the N-terminus of HuABC77D heavy chain does not negatively affect antigen binding of HuABC77D.

### Sequence Listing

Sequence listing
Amino acid sequence of HuABC2 VH
SEQ ID NO: 1
MKLWLNWVFLLTLLHGIQCEVQLVESGGGLVQPGGSLRLSCAASGFTFSDFYMEWVRQAPGKGLEWIAASRNKAND
YTTEYSASVKGRFIVSRDDSKNSLYLQMNSLKTEDTAVYYCARSYYRYDGMDYWGQGTTVTVSS
Amino acid sequence of HuABC2 VL
SEQ ID NO: 2
MDFQVQIFSFLLISASVIVSRGEIVLTQSPATLSLSPGERATLSCSAISSVSYMYWYQQKPGQAPRLLIYDTSNLVSGVPAR
FSGSGSGTDYTLTISSLEPEDFAVYYCQQWNTYPYTFGGGTKVEIK
Amino acid sequence of the CH1 region of human gamma-1 heavy chain encoded in
pHuABC2p
SEQ ID NO: 3
ASTKGPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKKV
Amino acid sequence of the hinge region of human gamma-1 heavy chain encoded in
pHuABC2p
SEQ ID NO: 4
EPKSCDKTHTCPPCP
Amino acid sequence of the CH2 region of human gamma-1 heavy chain encoded in
pHuABC2p
SEQ ID NO: 5
APELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAK
Amino acid sequence of the CH3 region of human gamma-1 heavy chain encoded in
pHuABC2p
SEQ ID NO: 6
GQPREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
Amino acid sequence of the Ck region encoded in pHuABC2p
SEQ ID NO: 7
TVAAPSVFIFPPSDEQLKSGTASVVCLLNNFYPREAKVQWKVDNALQSGNSQESVTEQDSKDSTYSLSSTLTLSKADYEK
HKVYACEVTHQGLSSPVTKSFNRGEC
Amino acid sequence of the mature heavy chain of HuABC2 encoded in pHuABC2p
SEQ ID NO: 8
EVQLVESGGGLVQPGGSLRLSCAASGFTFSDFYMEWVRQAPGKGLEWIAASRNKANDYTTEYSASVKGRFIVSRDDSK
NSLYLQMNSLKTEDTAVYYCARSYYRYDGMDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
Amino acid sequence of the mature light chain of HuABC2 encoded in pHuABC2p
SEQ ID NO: 9
EIVLTQSPATLSLSPGERATLSCSAISSVSYMYWYQQKPGQAPRLLIYDTSNLVSGVPARFSGSGSGTDYTLTISSLEPEDF
AVYYCQQWNTYPYTFGGGTKVEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Amino acid sequence of the extracellular domain of human CTLA-4
SEQ ID NO: 10
MHVAQPAVVLASSRGIASFVCEYASPGKATEVRVTVLRQADSQVTEVCAATYMMGNELTFLDDSICTGTSSGNQVNLT
IQGLRAMDTGLYICKVELMYPPPYYLGIGNGTQIYVIDPEPCPDSD
Amino acid sequence of the flexible polypeptide linker
SEQ ID NO: 11
SGGGGSG
Amino acid sequence of the fusion of CH3 to the linker and extracellular domain
of CTLA-4 (CH3-CTLA4 fusion) encoded in pHC624
SEQ ID NO: 12
GQPREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGSGGGGSGMHVAQPAVVLASSRGIASFVCEYASPGKATEVRVTVLRQADSQ
VTEVCAATYMMGNELTFLDDSICTGTSSGNQVNLTIQGLRAMDTGLYICKVELMYPPPYYLGIGNGTQIYVIDPEPCPDSD
Amino acid sequence of the mature heavy chain of HuABC2 fused at the C-terminus
to the linker followed by the extracellular domain of CTLA-4 encoded in pHC624
SEQ ID NO: 13
EVQLVESGGGLVQPGGSLRLSCAASGFTFSDFYMEWVRQAPGKGLEWIAASRNKANDYTTEYSASVKGRFIVSRDDSK
NSLYLQMNSLKTEDTAVYYCARSYYRYDGMDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGSGGGGSGMHVAQPAVVLASSR
GIASFVCEYASPGKATEVRVTVLRQADSQVTEVCAATYMMGNELTFLDDSICTGTSSGNQVNLTIQGLRAMDTGLYICK
VELMYPPPYYLGIGNGTQIYVIDPEPCPDSD
Amino acid sequence of the extracellular domain of the variant human CTLA-4
carrying the A29Y and L104E mutations (variant CTLA-4)
SEQ ID NO: 14
MHVAQPAVVLASSRGIASFVCEYASPGKYTEVRVTVLRQADSQVTEVCAATYMMGNELTFLDDSICTGTSSGNQVNLT
IQGLRAMDTGLYICKVELMYPPPYYEGIGNGTQIYVIDPEPCPDSD
Amino acid sequence of CH3 fused at the C-terminus to the linker followed by the
extracellular domain of the variant CTLA-4 encoded in pHC625 and pHC622
SEQ ID NO: 15
GQPREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGSGGGGSGMHVAQPAVVLASSRGIASFVCEYASPGKYTEVRVTVLRQADSQ
VTEVCAATYMMGNELTFLDDSICTGTSSGNQVNLTIQGLRAMDTGLYICKVELMYPPPYYEGIGNGTQIYVIDPEPCPDSD
Amino acid sequence of the mature heavy chain of HuABC2 fused at the C-terminus to
the linker followed by the extracellular domain of the variant CTLA-4 encoded in
pHC625 and pHC622
SEQ ID NO: 16
EVQLVESGGGLVQPGGSLRLSCAASGFTFSDFYMEWVRQAPGKGLEWIAASRNKANDYTTEYSASVKGRFIVSRDDSK
NSLYLQMNSLKTEDTAVYYCARSYYRYDGMDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGSGGGGSGMHVAQPAVVLASSR
GIASFVCEYASPGKYTEVRVTVLRQADSQVTEVCAATYMMGNELTFLDDSICTGTSSGNQVNLTIQGLRAMDTGLYICK
VELMYPPPYYEGIGNGTQIYVIDPEPCPDSD
ABC2 VH CDR1 amino acid sequence
SEQ ID NO: 17
DFYME
ABC2 VH CDR2 amino acid sequence
SEQ ID NO: 18
ASRNKANDYTTEYSASVKG
ABC2 VH CDR3 amino acid sequence
SEQ ID NO: 19
SYYRYDGMDY
ABC2 VL CDR1 amino acid sequence
SEQ ID NO: 20
SAISSVSYMY
ABC2 VL CDR2 amino acid sequence
SEQ ID NO: 21
DTSNLVS
ABC2 VL CDR3 amino acid sequence
SEQ ID NO: 22
QQWNTYPYT
ABC101 VH CDR1 amino acid sequence
SEQ ID NO: 23
NDNHWWN
ABC101 VH CDR2 amino acid sequence
SEQ ID NO: 24
YIDSSGSSDNNPSLKS
ABC101 VH CDR3 amino acid sequence
SEQ ID NO: 25
GGGRDYYGMDY
ABC101 VL CDR1 amino acid sequence
SEQ ID NO: 26
RASQSVSTSSYSYVH
ABC101 VL CDR2 amino acid sequence
SEQ ID NO: 27
YASNLES
ABC101 VL CDR3 amino acid sequence
SEQ ID NO: 28
QHSWDIPFT
Amino acid sequence of the mature HuABC101 VH
SEQ ID NO: 29
QVQLQESGPGLVKPSQTLSLTCTVSGYSITNDNHWWNWIRQHPGKGLEWMGYIDSSGSSDNNPSLKSQITISRDTSKN
QLSLKLSSVTAADTAVYYCARGGGRDYYGMDYWGQGTTVTVSS
Amino acid sequence of the mature HuABC101 VL
SEQ ID NO: 30
DIVMTQSPDSLGVSLGERATINCRASQSVSTSSYSYVHWYQQKPGQPPKLLIKYASNLESGVPDRFSGSGSGTDFTLTISS
LQAEDVAVYYCQHSWDIPFTFGQGTKLEIK
Amino acid sequence of the mature human CD122
SEQ ID NO: 31
AVNGTSQFTCFYNSRANISCVWSQDGALQDTSCQVHAWPDRRRWNQTCELLPVSQASWACNLILGAPDSQKLTTVD
IVTLRVLCREGVRWRVMAIQDFKPFENLRLMAPISLQVVHVETHRCNISWEISQASHYFERHLEFEARTLSPGHTWEEA
PLLTLKQKQEWICLETLTPDTQYEFQVRVKPLQGEFTTWSPWSQPLAFRTKPAALGKDTIPWLGHLLVGLSGAFGElILV
YLLINCRNTGPWLKKVLKCNTPDPSKFFSQLSSEHGGDVQKWLSSPFPSSSFSPGGLAPEISPLEVLERDKVTQLLLQQDK
VPEPASLSSNHSLTSCFTNQGYFFFHLPDALEIEACQVYFTYDPYSEEDPDEGVAGAPTGSSPQPLQPLSGEDDAYCTFPS
RDDLLLFSPSLLGGPSPPSTAPGGSGAGEERMPPSLQERVPRDWDPQPLGPPTPGVPDLVDFQPPPELVLREAGEEVP
DAGPREGVSFPWSRPPGQGEFRALNARLPLNTDAYLSLQELQGQDPTHLV
Amino acid sequence of Ck fused to the linker followed by the extracellular
domain of the variant CTLA-4 (Ck-CTLA4YE fusion) encoded in pHC623
SEQ ID NO: 32
RTVAAPSVFIFPPSDEQLKSGTASVVCLLNNFYPREAKVQWKVDNALQSGNSQESVTEQDSKDSTYSLSSTLTLSKADYE
KHKVYACEVTHQGLSSPVTKSFNRGECSGGGGSGMHVAQPAVVLASSRGIASFVCEYASPGKYTEVRVTVLRQADSQV
TEVCAATYMMGNELTFLDDSICTGTSSGNQVNLTIQGLRAMDTGLYICKVELMYPPPYYEGIGNGTQIYVIDPEPCPDSD
Amino acid sequence of the mature heavy chain of HuABC2 encoded in pHC623
SEQ ID NO: 33
EVQLVESGGGLVQPGGSLRLSCAASGFTFSDFYMEWVRQAPGKGLEWIAASRNKANDYTTEYSASVKGRFIVSRDDSK
NSLYLQMNSLKTEDTAVYYCARSYYRYDGMDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
Amino acid sequence of the mature light chain of HuABC2 fused at the C-terminus
to the linker followed by the extracellular domain of the variant CTLA-4 encoded
in pHC623
SEQ ID NO: 34
EIVLTQSPATLSLSPGERATLSCSAISSVSYMYWYQQKPGQAPRLLIYDTSNLVSGVPARFSGSGSGTDYTLTISSLEPEDF
AVYYCQQWNTYPYTFGGGTKVEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECSGGGGSGMHVAQPAVVLASSRGIASF
VCEYASPGKYTEVRVTVLRQADSQVTEVCAATYMMGNELTFLDDSICTGTSSGNQVNLTIQGLRAMDTGLYICKVELM
YPPPYYEGIGNGTQIYVIDPEPCPDSD
Amino acid sequence of the synthetic signal peptide
SEQ ID NO: 35
MGWSWIFFFLLSGTASVLS
Amino acid sequence of the flexible polypeptide linker
SEQ ID NO: 36
SGGGGSGGGGS
Amino acid sequence of the mature HuABC2 VH
SEQ ID NO: 37
EVQLVESGGGLVQPGGSLRLSCAASGFTFSDFYMEWVRQAPGKGLEWIAASRNKANDYTTEYSASVKGRFIVSRDDSK
NSLYLQMNSLKTEDTAVYYCARSYYRYDGMDYWGQGTTVTVSS
Amino acid sequence of the mature HuABC2 VH fused at the N-terminus to the signal
peptide followed by the linker and extracellular domain of the variant CTLA-4
(CTLA4YE-VH fusion)
SEQ ID NO: 38
MGWSWIFFFLLSGTASVLSMHVAQPAVVLASSRGIASFVCEYASPGKYTEVRVTVLRQADSQVTEVCAATYMMGNEL
TFLDDSICTGTSSGNQVNLTIQGLRAMDTGLYICKVELMYPPPYYEGIGNGTQIYVIDPEPCPDSDSGGGGSGGGGSEV
QLVESGGGLVQPGGSLRLSCAASGFTFSDFYMEWVRQAPGKGLEWIAASRNKANDYTTEYSASVKGRFIVSRDDSKNS
LYLQMNSLKTEDTAVYYCARSYYRYDGMDYWGQGTTVTVSS
Amino acid sequence of the mature heavy chain of HuABC2 fused at the N-terminus
to the extracellular domain of the variant CTLA-4 followed by the linker encoded
in pHC640
SEQ ID NO: 39
MHVAQPAVVLASSRGIASFVCEYASPGKYTEVRVTVLRQADSQVTEVCAATYMMGNELTFLDDSICTGTSSGNQVNLT
IQGLRAMDTGLYICKVELMYPPPYYEGIGNGTQIYVIDPEPCPDSDSGGGGSGGGGSEVQLVESGGGLVQPGGSLRLSC
AASGFTFSDFYMEWVRQAPGKGLEWIAASRNKANDYTTEYSASVKGRFIVSRDDSKNSLYLQMNSLKTEDTAVYYCAR
SYYRYDGMDYWGQGTTVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEAAGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Amino acid sequence of the mature HuABC2 VL
SEQ ID NO: 40
EIVLTQSPATLSLSPGERATLSCSAISSVSYMYWYQQKPGQAPRLLIYDTSNLVSGVPARFSGSGSGTDYTLTISSLEPEDF
AVYYCQQWNTYPYTFGGGTKVEIK
Amino acid sequence of the mature HuABC2 VL fused at the N-terminus to the
extracellular domain of the variant CTLA-4 followed by the linker (CTLA4YE-Vk
fusion)
SEQ ID NO: 41
MHVAQPAVVLASSRGIASFVCEYASPGKYTEVRVTVLRQADSQVTEVCAATYMMGNELTFLDDSICTGTSSGNQVNLT
IQGLRAMDTGLYICKVELMYPPPYYEGIGNGTQIYVIDPEPCPDSDSGGGGSGGGGSEIVLTQSPATLSLSPGERATLSCS
AISSVSYMYWYQQKPGQAPRLLIYDTSNLVSGVPARFSGSGSGTDYTLTISSLEPEDFAVYYCQQWNTYPYTFGGGTKVEIK
Amino acid sequence of the mature light chain of HuABC2 fused at the N-terminus
to the extracellular domain of the variant CTLA-4 followed by the linker encoded
in pHC641
SEQ ID NO: 42
MHVAQPAVVLASSRGIASFVCEYASPGKYTEVRVTVLRQADSQVTEVCAATYMMGNELTFLDDSICTGTSSGNQVNLT
IQGLRAMDTGLYICKVELMYPPPYYEGIGNGTQIYVIDPEPCPDSDSGGGGSGGGGSEIVLTQSPATLSLSPGERATLSCS
AISSVSYMYWYQQKPGQAPRLLIYDTSNLVSGVPARFSGSGSGTDYTLTISSLEPEDFAVYYCQQWNTYPYTFGGGTKV
EIKRTVAAPSVFIFPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC
Amino acid sequence of the extra cellular region of human CD80
SEQ ID NO: 43
VIHVTKEVKEVATLSCGHNVSVEELAQTRIYWQKEKKMVLTMMSGDMNIWPEYKNRTIFDITNNLSIVILALRPSDEGT
YECVVLKYEKDAFKREHLAEVTLSVKADFPTPSISDFEIPTSNIRRIICSTSGGFPEPHLSWLENGEELNAINTTVSQDPETEL
YAVSSKLDFNMTTNHSFMCLIKYGHLRVNQTFNWNTTKQEHFPDN
Amino acid sequence of the FLAG polypeptide
SEQ ID NO: 44
DYKDDDDK
Amino acid sequence of the GPI anchorage signal derived from human CD55
SEQ ID NO: 45
PNKGSGTTSGTTRLLSGHTCFTLTGLLGTLVTMGLLT
Amino acid sequence of the mature CD80-FLAG-GPI
SEQ ID NO: 46
VIHVTKEVKEVATLSCGHNVSVEELAQTRIYWQKEKKMVLTMMSGDMNIWPEYKNRTIFDITNNLSIVILALRPSDEGT
YECVVLKYEKDAFKREHLAEVTLSVKADFPTPSISDFEIPTSNIRRIICSTSGGFPEPHLSWLENGEELNAINTTVSQDPETEL
YAVSSKLDFNMTTNHSFMCLIKYGHLRVNQTFNWNTTKQEHFPDNTGGGDYKDDDDKGGGPNKGSGTTSGTTRLLS
GHTCFTLTGLLGTLVTMGLLT
Amino acid sequence of the extra cellular region of human CD86
SEQ ID NO: 47
LKIQAYFNETADLPCQFANSQNQSLSELVVFWQDQENLVLNEVYLGKEKFDSVHSKYMGRTSFDSDSWTLRLHNLQIK
DKGLYQCIIHHKKPTGMIRIHQMNSELSVLANFSQPEIVPISNITENVYINLTCSSIHGYPEPKKMSVLLRTKNSTIEYDGIM
QKSQDNVTELYDVSISLSVSFPDVTSNMTIFCILETDKTRLLSSPFSIELEDPQPPPDH
Amino acid sequence of the mature CD86-FLAG-GPI
SEQ ID NO: 48
LKIQAYFNETADLPCQFANSQNQSLSELVVFWQDQENLVLNEVYLGKEKFDSVHSKYMGRTSFDSDSWTLRLHNLQIK
DKGLYQCIIHHKKPTGMIRIHQMNSELSVLANFSQPEIVPISNITENVYINLTCSSIHGYPEPKKMSVLLRTKNSTIEYDGIM
QKSQDNVTELYDVSISLSVSFPDVTSNMTIFCILETDKTRLLSSPFSIELEDPQPPPDHTGGGDYKDDDDKGGGPNKGSG
TTSGTTRLLSGHTCFTLTGLLGTLVTMGLLT
Amino acid sequence of belatacept
SEQ ID NO: 49
MHVAQPAVVLASSRGIASFVCEYASPGKYTEVRVTVLRQADSQVTEVCAATYMMGNELTFLDDSICTGTSSGNQVNLT
IQGLRAMDTGLYICKVELMYPPPYYEGIGNGTQIYVIDPEPCPDSDQEPKSSDKTHTSPPSPAPELLGGSSVFLFPPKPKD
TLMISRTPEVTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
Amino acid sequence of the polypeptide linker
SEQ ID NO: 50
SGGGGSGGGGSGGGGS
Amino acid sequence of the extracellular domain of human CD122
SEQ ID NO: 51
AVNGTSQFTCFYNSRANISCVWSQDGALQDTSCQVHAWPDRRRWNQTCELLPVSQASWACNLILGAPDSQKLTTVD
IVTLRVLCREGVRWRVMAIQDFKPFENLRLMAPISLQVVHVETHRCNISWEISQASHYFERHLEFEARTLSPGHTWEEA
PLLTLKQKQEWICLETLTPDTQYEFQVRVKPLQGEFTTWSPWSQPLAFRTKPAALGKDT
Amino acid sequence of CD122-FLAG-GPI encoded in pFCm148
SEQ ID NO: 52
AVNGTSQFTCFYNSRANISCVWSQDGALQDTSCQVHAWPDRRRWNQTCELLPVSQASWACNLILGAPDSQKLTTVD
IVTLRVLCREGVRWRVMAIQDFKPFENLRLMAPISLQVVHVETHRCNISWEISQASHYFERHLEFEARTLSPGHTWEEA
PLLTLKQKQEWICLETLTPDTQYEFQVRVKPLQGEFTTWSPWSQPLAFRTKPAALGKDTTGGGDYKDDDDKGGGPNK
GSGTTSGTTRLLSGHTCFTLTGLLGTLVTMGLLT
Amino acid sequence of the mature heavy chain of HuABC101 encoded in pHuABC101
SEQ ID NO: 53
QVQLQESGPGLVKPSQTLSLTCTVSGYSITNDNHWWNWIRQHPGKGLEWMGYIDSSGSSDNNPSLKSQITISRDTSKN
QLSLKLSSVTAADTAVYYCARGGGRDYYGMDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
Amino acid sequence of the mature light chain of HuABC101 encoded in pHuABC101
SEQ ID NO: 54
DIVMTQSPDSLGVSLGERATINCRASQSVSTSSYSYVHWYQQKPGQPPKLLIKYASNLESGVPDRFSGSGSGTDFTLTISS
LQAEDVAVYYCQHSWDIPFTFGQGTKLEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
Amino acid sequence of the mature HuABC101 VH fused at the N-terminus to the
extracellular domain of the variant CTLA-4 followed by the linker (CTLA4YE-
HuABC101 VH fusion)
SEQ ID NO: 55
MGWSWIFFFLLSGTASVLSMHVAQPAVVLASSRGIASFVCEYASPGKYTEVRVTVLRQADSQVTEVCAATYMMGNEL
TFLDDSICTGTSSGNQVNLTIQGLRAMDTGLYICKVELMYPPPYYEGIGNGTQIYVIDPEPCPDSDSGGGGSGGGGSQV
QLQESGPGLVKPSQTLSLTCTVSGYSITNDNHWWNWIRQHPGKGLEWMGYIDSSGSSDNNPSLKSQITISRDTSKNQL
SLKLSSVTAADTAVYYCARGGGRDYYGMDYWGQGTTVTVSS
Amino acid sequence of the mature heavy chain of HuABC101 fused at the N-terminus
to the extracellular domain of the variant CTLA-4 followed by the linker encoded
in pHC642
SEQ ID NO: 56
MHVAQPAVVLASSRGIASFVCEYASPGKYTEVRVTVLRQADSQVTEVCAATYMMGNELTFLDDSICTGTSSGNQVNLT
IQGLRAMDTGLYICKVELMYPPPYYEGIGNGTQIYVIDPEPCPDSDSGGGGSGGGGSQVQLQESGPGLVKPSQTLSLTC
TVSGYSITNDNHWWNWIRQHPGKGLEWMGYIDSSGSSDNNPSLKSQITISRDTSKNQLSLKLSSVTAADTAVYYCARG
GGRDYYGMDYWGQGTTVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Amino acid sequence of the mature heavy chain of HuABC101 fused at the C-terminus
to the linker followed by the extracellular domain of the variant CTLA-4 encoded
in pHC630
SEQ ID NO: 57
QVQLQESGPGLVKPSQTLSLTCTVSGYSITNDNHWWNWIRQHPGKGLEWMGYIDSSGSSDNNPSLKSQITISRDTSKN
QLSLKLSSVTAADTAVYYCARGGGRDYYGMDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGSGGGGSGMHVAQPAVVLASSRGI
ASFVCEYASPGKYTEVRVTVLRQADSQVTEVCAATYMMGNELTFLDDSICTGTSSGNQVNLTIQGLRAMDTGLYICKVE
LMYPPPYYEGIGNGTQIYVIDPEPCPDSD
Amino acid sequence of HuABC77D VH
SEQ ID NO: 58
MDFGLIFFIVALLKGVQCEVQLVESGGGLVQPGGSLRLSCAASGFDFSRYWMSWVRQAPGKGLEWIGEINPDSSTINY
APSLKDRFTISRDNAKNSLYLQMNSLRAEDTAVYYCARHDYDEGIYFDYWGQGTTVTVSS
HuABC77D VH CDR1 amino acid sequences
SEQ ID NO: 59
RYWMS
HuABC77D VH CDR2 amino acid sequences
SEQ ID NO: 60
EINPDSSTINYAPSLKD
HuABC77D VH CDR3 amino acid sequences
SEQ ID NO: 61
HDYDEGIYFDY
Amino acid sequence of HuABC77D VL
SEQ ID NO: 62
MDFQVQIFSFLLISASVIISRGDIQMTQSPSSLSASVGDRVTITCSASSRVIYMHWYQQKPGKAPKRWIYDTSKLASGVPS
RFSGSGSGTEFTLTISSLQPEDFATYYCQQWSSNPPTFGGGTKVEIK
HuABC77D VL CDR1 amino acid sequences
SEQ ID NO: 63
SASSRVIYMH
HuABC77D VL CDR2 amino acid sequences
SEQ ID NO: 64
DTSKLAS
HuABC77D VL CDR3 amino acid sequences
SEQ ID NO: 65
QQWSSNPPT
Amino acid sequence of the mature heavy chain of HuABC77D encoded in pHuABC77D
SEQ ID NO: 66
EVQLVESGGGLVQPGGSLRLSCAASGFDFSRYWMSWVRQAPGKGLEWIGEINPDSSTINYAPSLKDRFTISRDNAKNS
LYLQMNSLRAEDTAVYYCARHDYDEGIYFDYWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
Amino acid sequence of the mature light chain of HuABC77D encoded in pHuABC77D
SEQ ID NO: 67
DIQMTQSPSSLSASVGDRVTITCSASSRVIYMHWYQQKPGKAPKRWIYDTSKLASGVPSRFSGSGSGTEFTLTISSLQPE
DFATYYCQQWSSNPPTFGGGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Amino acid sequence of the mature HuABC77D VH
SEQ ID NO: 68
EVQLVESGGGLVQPGGSLRLSCAASGFDFSRYWMSWVRQAPGKGLEWIGEINPDSSTINYAPSLKDRFTISRDNAKNS
LYLQMNSLRAEDTAVYYCARHDYDEGIYFDYWGQGTTVTVSS
Amino acid sequence of the mature HuABC77D VH fused at the N-terminus to the
extracellular domain of the variant CTLA-4 followed by the linker (CTLA4YE-
HuABC77D VH fusion)
SEQ ID NO: 69
MHVAQPAVVLASSRGIASFVCEYASPGKYTEVRVTVLRQADSQVTEVCAATYMMGNELTFLDDSICTGTSSGNQVNLT
IQGLRAMDTGLYICKVELMYPPPYYEGIGNGTQIYVIDPEPCPDSDSGGGGSGGGGSEVQLVESGGGLVQPGGSLRLSC
AASGFDFSRYWMSWVRQAPGKGLEWIGEINPDSSTINYAPSLKDRFTISRDNAKNSLYLQMNSLRAEDTAVYYCARHD
YDEGIYFDYWGQGTTVTVSS
Amino acid sequence of the mature heavy chain of HuABC77D fused at the N-terminus
to the extracellular domain of the variant CTLA-4 followed by the linker encoded
in pHC644
SEQ ID NO: 70
MHVAQPAVVLASSRGIASFVCEYASPGKYTEVRVTVLRQADSQVTEVCAATYMMGNELTFLDDSICTGTSSGNQVNLT
IQGLRAMDTGLYICKVELMYPPPYYEGIGNGTQIYVIDPEPCPDSDSGGGGSGGGGSEVQLVESGGGLVQPGGSLRLSC
AASGFDFSRYWMSWVRQAPGKGLEWIGEINPDSSTINYAPSLKDRFTISRDNAKNSLYLQMNSLRAEDTAVYYCARHD
YDEGIYFDYWGQGTTVTVSSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGK
Amino acid sequence of the mature heavy chain of HuABC77D fused at the C-terminus
to the linker followed by the extracellular domain of the variant CTLA-4 encoded
in pHC645
SEQ ID NO: 71
EVQLVESGGGLVQPGGSLRLSCAASGFDFSRYWMSWVRQAPGKGLEWIGEINPDSSTINYAPSLKDRFTISRDNAKNS
LYLQMNSLRAEDTAVYYCARHDYDEGIYFDYWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGSGGGGSGMHVAQPAVVLASSRGI
ASFVCEYASPGKYTEVRVTVLRQADSQVTEVCAATYMMGNELTFLDDSICTGTSSGNQVNLTIQGLRAMDTGLYICKVE
LMYPPPYYEGIGNGTQIYVIDPEPCPDSD

1-38. (canceled)

39. A bifunctional molecule, comprising a mature heavy chain comprising a heavy chain variable region of SEQ ID NO:37 linked at its C-terminus to a heavy chain human IgG constant region and at its N-terminus via a polypeptide linker to a CTLA-4 extracellular domain having at least 90% sequence identity to SEQ ID NO:10, and a mature light chain comprising SEQ ID NO:9.

40. The bifunctional molecule of claim 39, wherein the mature heavy chain comprises SEQ ID NO:39 provided the constant region of the mature heavy chain can be mutated to increase FcRn binding and/or decrease effector function, and the C-terminal lysine can be omitted.

41. The bifunctional molecule of claim 40, wherein the constant region includes glutamine at position 250 and/or leucine at position 428 by EU numbering to increase FcRn binding.

42. The bifunctional molecule of claim 40, wherein the constant region includes alanine at position 234 and alanine at position 235 by EU numbering to decrease effector function.

43. The bifunctional molecule of claim 39, wherein the polypeptide linker comprises the amino acid sequence of SEQ ID NO:11, 36 or 50.

44. The bifunctional molecule of claim 39, in the form of a heterodimer comprising two copies of the heavy chain and two copies of the light chain.

45. The bifunctional molecule of claim 39, wherein the extracellular domain of CTLA-4 comprises SEQ ID NO:10.

46. The bifunctional molecule of claim 39, wherein the extracellular domain of

47. A pharmaceutical composition comprising a bifunctional molecule of claim 39 and a pharmaceutically acceptable carrier.

48. A method of treating a subject having an immune disorder, comprising administering an effective regime of a bifunctional molecule of claim 39 to the subject.

49. The method of claim 48, wherein the immune disorder is any of systemic lupus erythematosus, vitiligo, alopecia areata, type 1 diabetes, celiac disease, non-alcoholic steatohepatitis, skin allograft rejection, multiple sclerosis, polymyositis, allergic dermatitis, primary biliary cirrhosis, Behcet's disease, and ulcerative colitis.
